SEQUENCE LISTING (17 sequences)

SEQ ID NO: 1 - protein, 243 aa, synthetic construct
  REGION 1..243: note = anti-HLA-A2 scFv antibody
DVVMTQSPSSLSASVGDRVTITCQASQDISNYLNWYQQKPGKAPKLLIYDASNLETGVPSRFSGSGSGTDFTFTISSLQPEDFATYYCQQYSSFPLTFGGGTKVDIKGGGGSGGGGSGGGGSQVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTLVTV

SEQ ID NO: 2 - protein, 468 aa, synthetic construct
  REGION 1..468: note = anti-HLA-A2 CAR
DVVMTQSPSSLSASVGDRVTITCQASQDISNYLNWYQQKPGKAPKLLIYDASNLETGVPSRFSGSGSGTDFTFTISSLQPEDFATYYCQQYSSFPLTFGGGTKVDIKGGGGSGGGGSGGGGSQVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTLVTVTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 118 aa, synthetic construct [sequence omitted]

SEQ ID NO: 4 - protein, 108 aa, synthetic construct [sequence omitted]

SEQ ID NO: 5 - protein, 119 aa, synthetic construct [sequence omitted]

SEQ ID NO: 6 - protein, 106 aa, synthetic construct [sequence omitted]

SEQ ID NO: 7 - protein, 119 aa, synthetic construct [sequence omitted]

SEQ ID NO: 8 - protein, 110 aa, synthetic construct [sequence omitted]

SEQ ID NO: 9 - protein, 119 aa, synthetic construct [sequence omitted]

SEQ ID NO: 10 - protein, 106 aa, synthetic construct [sequence omitted]

SEQ ID NO: 11 - protein, 120 aa, synthetic construct [sequence omitted]

SEQ ID NO: 12 - protein, 106 aa, synthetic construct [sequence omitted]

SEQ ID NO: 13 - protein, 213 aa, synthetic construct
  REGION 1..213: note = Muromonab-CD3 light chain
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRADTAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 14 - protein, 450 aa, synthetic construct
  REGION 1..450: note = Muromonab-CD3 heavy chain
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAKTTAPSVYPLAPVCGGTTGSSVTLGCLVKGYFPEPVTLTWNSGSLSSGVHTFPAVLQSDLYTLSSSVTVTSSTWPSQSITCNVAHPASSTKVDKKIEPRPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 213 aa, synthetic construct
  REGION 1..213: note = teplizumab light chain
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 449 aa, synthetic construct
  REGION 1..449: note = teplizumab heavy chain
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - DNA, 23 bp, synthetic construct
  misc_feature 1..23: note = TRAC CRISPR RNA (crRNA)-targeting sequence
  source 1..23: mol_type = other DNA
ctctcagctggtacacggcaggg

(19) United States
(12) Patent Application Publication
(10) Pub. No.: US 2025/0099584 A1
(43) Pub. Date: Mar. 27, 2025

(54) USE OF TCR-DEFICIENT CAR-TREGS IN COMBINATION WITH ANTI-TCR COMPLEX MONOCLONAL ANTIBODIES FOR INDUCING DURABLE TOLERANCE

(71) Applicants: INSTITUT NATIONAL DE LA SANTÉ ET DE LA RECHERCHE MÉDICALE, Paris (FR); UNIVERSITÉ PARIS CITÉ, Paris (FR); FONDATION IMAGINE, Paris (FR); ASSISTANCE PUBLIQUE-HÔPITAUX DE PARIS (APHP), Paris (FR)

(72) Inventors: Julien ZUBER, Paris (FR); Tifanie BLEIN, Paris (FR); Soëli CHARBONNIER, Paris (FR)

(21) Appl. No.: 18/728,994

(22) PCT Filed: Feb. 17, 2023
(86) PCT No.: PCT/EP2023/054017

§ 371 Date: Jul. 15, 2024

(30) Foreign Application Priority Data
Feb. 18, 2022 (EP) 22305182.2

(51) Int. Cl. A61K 39/00; A61K 39/395; A61P 37/06; C12N 9/22; C12N 15/11; C12N 15/90

(52) U.S. Cl. CPC A61K 40/11; A61K 39/3955; A61K 40/31; A61K 40/416; A61K 40/418; A61P 37/06; C12N 9/22; C12N 15/11; C12N 15/907; C12N 2310/20

(57) ABSTRACT

The present invention is defined by the claims. In particular, the present invention relates to the use of TCR-deficient CAR-Tregs in combination with anti-TCR complex monoclonal antibodies for inducing durable tolerance.

### FIELD OF THE INVENTION

The present invention is in the field of medicine, in particular immunology.

### BACKGROUND OF THE INVENTION

Transplantation is one of the most challenging and complex areas of medicine, and involves the transfer of a tissue or organ from a donor to a recipient patient. It offers the possibility to replace the recipient's damaged or defective tissue or organ with a functional one and can significantly improve the health and well-being of the recipient. Organ transplantation has undergone substantial improvements in both the prevention and treatment of acute rejection, but subclinical episodes and chronic graft dysfunction still heavily impact medium- and long-term graft survival.

Rejection arises due to sensitisation of the cell-mediated immune system of the recipient to the foreign (allogeneic) antigens of the donor. In particular, the recipient's immune system reacts to the major histocompatibility complex (MHC) molecules presented on the surface of the donor tissues (the graft). The MHC molecules are expressed on the surface of cells in all jawed vertebrates, and are responsible for displaying antigens to cytotoxic T cells. MHC molecules also contribute to the risk of graft versus host disease (GVHD) after haematopoietic stem cell transplantation. Matching of MHC class I and II genes is essential for the success of transplantation. Graft-versus-host disease (GVHD) is associated with significant morbidity and mortality. Mortality rates as a direct or indirect consequence of GVHD can reach 50% despite the prophylactic use of immunsuppressive drugs like cyclosporine, tacrolimus, ATG, methotrexate, and mycophenolate mofetil which are administered for prevention of GVHD. In humans, genes encoding for MHC molecules are referred to as human leukocyte antigen (HLA) genes. The most intensely studied HLA genes are the nine so-called classical MHC genes: HLA-A, HLA-B, HLA-C, HLA-DPA 1, HLA-DPB 1, HLA-DQA 1, HLA-DQB 1, HLA-DRA, and HLA-DRB 1. In humans, the MHC is divided into three regions: Class I, II, and III. The A, B, and C genes belong to MHC class I, whereas the six D genes belong to class II. Immunological rejection is typically alleviated by administering immunosuppressant drugs to the recipient, both prior to and after the transplantation. Immunosuppressants decrease the activity of the recipient's immune system, thereby preventing it from attacking the donor tissue or organ and thus allowing better graft retention. However, the administration of immunosuppressants does not result in the patient developing long-term tolerance to the allograft, and, therefore, most patients must undergo immunosuppressive therapy for the lifetime of the graft (typically 5-10 years) or the remainder of their lives. Furthermore immunosuppressants are known to cause a number of complications, largely owing to their non-specificity. Emerging therapeutic strategies, among them induction of tolerance to donor antigens, are moving to the clinical stage after years of experimental model work. Among natural mechanisms and tolerance-inductive strategies, the uses of different types of regulatory cells are among the most promising ones. In particular, the uses of CD4+ and CD8+ Tregs have been highlighted in recent years. However, polyclonal Tregs encompass many specificities and could potentially be globally immunosuppressive. Thus, the use of antigen-specific Tregs seems to be preferable for the next generation of Treg therapeutic approaches.

An attractive alternative would be engineering recipient Tregs with a chimeric antigen receptor (CAR) that redirects their antigen specificity toward a given donor antigen. CARs are engineered receptors that consist of an antigen-binding ectodomain, a hinge region linked to the transmembrane domain, and an intracyctoplasmic domain, commonly composed of one or several costimulation domains (CSDs), and CD3ζ signalling tail. The antigen-binding motif is frequently a single-chain variable fragment (ScFv) that combines the heavy and light chains of an antibody. CARs are referred to as first-, second-, and third-generation depending on the number of incorporated CSDs (none, one or several, respectively). Second generation CARs, including either the CD28 or 4-1BB CSD, have been the most broadly used in the clinic.

Although CAR technology has been successfully translated to clinical oncology and extensively refined to further improve the efficacy-safety balance, its use in human Tregs is still an emerging field of investigation. In particular, human leukocyte antigen (HLA) A2-targeted CAR-Tregs efficiently prevent graft-versus-host disease (GVHD) and skin graft rejection in an HLA-A2-specific manner (Dawson, N. A. J. et al. Functional effects of chimeric antigen receptor co-receptor signalling domains in human regulatory T cells. Sci. Transl. Med. 12, eaaz3866 (2020)). However, in conventional T cells (Tconvs), CAR expression is often associated with tonic signalling, which can induce CAR-T cell dysfunction. The extent and effects of CAR tonic signalling vary greatly according to the expression intensity and intrinsic properties of the CAR. Recently, it was shown that the 4-1BB CSD-associated tonic signal yields a more dramatic effect in CAR-Tregs than in CAR-Tconvs with respect to activation and proliferation (Lamarthee B, Marchal A, Charbonnier S, Blein T, Leon J, Martin E, Rabaux L, Vogt K, Titeux M, Delville M, Vinçon H, Six E, Pallet N, Michonneau D, Anglicheau D, Legendre C, Taupin J L, Nemazanyy I, Sawitzki B, Latour S, Cavazzana M, André I, Zuber J. Transient mTOR inhibition rescues 4-1BB CAR-Tregs from tonic signal-induced dysfunction. Nat Commun. 2021 Nov. 8;12 (1): 6446. doi: 10.1038/s41467-021-26844-1. PMID: 34750385; PMCID: PMC8575891). Compared to CD28 CAR-Tregs, 4-1BB CAR-Tregs exhibit decreased lineage stability and reduced in vivo suppressive capacities. Transient exposure of 4-1BB CAR-Tregs to a Treg stabilizing cocktail, including an mTOR inhibitor and vitamin C, during ex vivo expansion sharply improves their in vivo function and expansion after adoptive transfer. This study demonstrates that the negative effects of 4-1BB tonic signalling in Tregs can be mitigated by transient mTOR inhibition.

Despite these considerable achievements, there is room for improving the efficacy of CAR-Tregs for the induction of a durable tolerance.

Anti-TCR complex monoclonal antibodies (mAbs), in particular anti-CD3 antibodies, effectively treat autoimmune disease in animal models and have also shown promise in clinical trials (Kuhn C, Weiner HL. Therapeutic anti-CD3 monoclonal antibodies: from bench to bedside. Immunotherapy. 2016 July; 8 (8): 889-906. doi: 10.2217/imt-2016-0049. Epub 2016 May 10. PMID: 27161438). Tolerance induction by anti-CD3 mAbs is related to the induction of Tregs that control pathogenic autoimmune responses. Binding of anti-CD3 mAb to the CD3/TCR complex indeed leads to antigenic modulation, i.e., disappearance of the CD3/TCR from the cells surface by shedding or internalization, rendering T cells blind toward their cognate antigen. At the same time anti-CD3 mAb-induced signalling through the CD3/TCR complex can render the T cell anergic or trigger apoptosis. However, the combination with CAR-Tregs has never been investigated.

### SUMMARY OF THE INVENTION

The present invention is defined by the claims. In particular, the present invention relates to the use of TCR-deficient CAR-Tregs in combination with anti-TCR complex monoclonal antibodies for inducing durable tolerance.

### DETAILED DESCRIPTION OF THE INVENTION

### Main Definitions

As used herein, the terms “polypeptide”, “peptide”, and “protein” are used interchangeably herein to refer to polymers of amino acids of any length. The terms also encompass an amino acid polymer that has been modified; for example, disulfide bond formation, glycosylation, lipidation, phosphorylation, or conjugation with a labeling component. Polypeptides when discussed in the context of gene therapy refer to the respective intact polypeptide, or any fragment or genetically engineered derivative thereof, which retains the desired biochemical function of the intact protein.

As used herein, the term “polynucleotide” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

As used herein, the expression “derived from” refers to a process whereby a first component (e.g., a first polypeptide), or information from that first component, is used to isolate, derive or make a different second component (e.g., a second polypeptide that is different from the first one).

As used herein, the “percent identity” between the two sequences is a function of the number of identical positions shared by the sequences (i.e., % identity=number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described below. The percent identity between two amino acid sequences can be determined using the Needleman and Wunsch algorithm (Needleman, Saul B. & Wunsch, Christian D. (1970). “A general method applicable to the search for similarities in the amino acid sequence of two proteins”. Journal of Molecular Biology. 48 (3): 443-53.). The percent identity between two nucleotide or amino acid sequences may also be determined using for example algorithms such as EMBOSS Needle (pair wise alignment; available at www.ebi.ac.uk). For example, EMBOSS Needle may be used with a BLOSUM62 matrix, a “gap open penalty” of 10, a “gap extend penalty” of 0.5, a false “end gap penalty”, an “end gap open penalty” of 10 and an “end gap extend penalty” of 0.5. In general, the “percent identity” is a function of the number of matching positions divided by the number of positions compared and multiplied by 100. For instance, if 6 out of 10 sequence positions are identical between the two compared sequences after alignment, then the identity is 60%. The % identity is typically determined over the whole length of the query sequence on which the analysis is performed. Two molecules having the same primary amino acid sequence or nucleic acid sequence are identical irrespective of any chemical and/or biological modification. According to the invention, a first amino acid sequence having at least 90% of identity with a second amino acid sequence means that the first sequence has 90; 91; 92; 93; 94; 95; 96; 97; 98; 99 or 100% of identity with the second amino acid sequence.

As used herein, the term “conservative sequence modifications” refers to amino acid modifications that do not significantly affect or alter the biologic function of the protein containing the amino acid sequence. Such conservative modifications include amino acid substitutions, additions and deletions. Modifications can be introduced into a protein by standard techniques known in the art, such as site-directed mutagenesis and PCR-mediated mutagenesis. A “conservative substitution” is one in which an amino acid is substituted for another amino acid that has similar properties, such that one skilled in the art of peptide chemistry would expect the secondary structure and hydropathic nature of the polypeptide to be substantially unchanged. Amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine. Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. Other families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

As used herein, the term “encoding” refers to the inherent property of specific sequences of nucleotides in a polynucleotide, such as, for example, a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “polynucleotide sequence encoding an amino acid sequence” includes all nucleotide sequences that are degenerate versions of each other and that encode the same amino acid sequence. The phrase “polynucleotide sequence that encodes a protein or a RNA” may also include introns to the extent that the nucleotide sequence encoding the protein may in some version contain an intron(s).

As used herein, the term “subject”, “host”, “individual” or “patient” refers to a mammal, preferably a human being, male or female at any age that is in-need of a therapy.

As used herein, the term “organ” refers to a solid vascularized organ that performs a specific function or group of functions within an organism. The term organ includes, but is not limited to, heart, lung, kidney, liver, pancreas, skin, uterus, bone, cartilage, small or large bowel, bladder, brain, breast, blood vessels, esophagus, fallopian tube, gallbladder, ovaries, pancreas, prostate, placenta, spinal cord, limb including upper and lower, spleen, stomach, testes, thymus, thyroid, trachea, ureter, urethra, uterus.

As used herein, the term “tissue” refers to any type of tissue in human or animals, and includes, but is not limited to, vascular tissue, skin tissue, hepatic tissue, pancreatic tissue, neural tissue, urogenital tissue, gastrointestinal tissue, skeletal tissue including bone and cartilage, adipose tissue, connective tissue including tendons and ligaments, amniotic tissue, chorionic tissue, dura, pericardia, muscle tissue, glandular tissue, facial tissue, ophthalmic tissue.

As used herein, the term “cell” refers to any eukaryotic cell. In some embodiments the cells are selected from the group consisting of multipotent hematopoietic stem cells derived from bone marrow, peripheral blood, or umbilical cord blood; or pluripotent (i.e. embryonic stem cells(ES) or induced pluripotent stem cells (iPS)) or multipotent stem cell-derived differentiated cells of different cell lineages such as cardiomyocytes, beta-pancreatic cells, hepatocytes, neurons, etc . . . .

As used herein, the term “population” refers to a population of cells, wherein the majority (e.g., at least about 50%, preferably at least about 60%, more preferably at least about 70%, and even more preferably at least about 80%) of the total number of cells have the specified characteristics of the cells of interest and express the markers of interest (e.g. a population of human CAR-Treg cells comprises at least about 50%, preferably at least about 60%, more preferably at least about 70%, and even more preferably at least about 80% of cells which have the highly suppressive functions and which express the particular markers of interest).

As used herein, the term “Treg” or “T regulatory cell” denotes a T lymphocyte endowed with a given antigen specificity imprinted by the TCR it expresses and with regulatory properties defined by the ability to suppress the response of conventional T lymphocytes or other immune cells. Such responses are known in the art and include, but are not limited to, cytotoxic activity against antigen-presenting target cells and secretion of different cytokines. Different types of Tregs exist and include, but are not limited to: inducible and thymic-derived Tregs, as characterized by different phenotypes such as CD4+CD25+/high, CD4+CD25+/highCD127-/low alone or in combination with additional markers that include, but are not limited to, FoxP3, neuropilin-1 (CD304), glucocorticoid-induced TNFR-related protein (GITR), cytotoxic T-lymphocyte-associated protein 4 (CTLA-4, CD152); T regulatory type 1 cells; T helper 3 cells. In some embodiments, the Treg is CD4+ Foxp3+ Treg cell or a CD8+ Foxp3+ Treg cells or a CD4+CD45RC^{low/-} Treg or a CD8+CD45RC^{low/-} cells or CD4+ Foxp3-Tr1 Tregs. All these Tregs can be transformed with the CAR of the present invention either upon direct ex vivo purification or upon in vitro expansion or differentiation from different precursor cells. Examples of in vitro amplification protocols can be found in Battaglia et al., J. Immunol. 177:8338-8347 (2006), Putnam et al., Diabetes 58:652-662 (2009), Gregori et al., Blood 116:935-944 (2009).

As used herein, the term “T-cell receptor” or “TCR” is used in the conventional sense to mean a molecule found on the surface of T cells that is responsible for recognizing antigens bound to MHC molecules. The molecule may be a heterodimer of two chains α and β (or optionally γ and δ) or it may be a recombinant single chain TCR construct. The variable domain of both the TCR α-chain and β-chain have three hypervariable or complementarity determining regions (CDRs). CDR3 is the main CDR responsible for recognizing processed antigen. Each chain of the TCR is thus a member of the immunoglobulin superfamily and possesses one N-terminal immunoglobulin (Ig)-variable (V) domain, one Ig-constant (C) domain, a transmembrane region, and a short cytoplasmic tail at the C-terminal end. The constant domain of the TCR consists of short connecting sequences in which a cysteine residue forms a disulfide bond, making a link between the two chains. The structure allows the TCR to associate with other molecules like CD3 which possess three distinct chains (γ, δ, and ε) in mammals and the ζ-chain. These accessory molecules have negatively charged transmembrane regions and are vital to propagating the signal from the TCR into the cell. The signal from the TCR complex is enhanced by simultaneous binding of the MHC molecules by a specific co-receptor. On helper T cells, this co-receptor is CD4 (specific for class II MHC); whereas on cytotoxic T cells, this co-receptor is CD8 (specific for class I MHC). The co-receptor not only ensures the specificity of the TCR for an antigen, but also allows prolonged engagement between the antigen presenting cell and the T cell and recruits essential molecules (e.g., LCK) inside the cell involved in the signalling of the activated T lymphocyte.

As used herein, the term “TRAC” refers to the T Cell Receptor Alpha Constant gene that encodes for the alpha chain to the TCR.

As used herein, the term “CD3” has its general meaning in the art and refers to the protein complex associated with the T cell receptor is composed of four distinct chains. In mammals, the complex contains a CD3γ chain, a CD3δ chain, and two CD3ε chains. These chains associate with the TCR and the (ζ-chain (zeta-chain) to generate an activation signal in T lymphocytes. The TCR, (ζ-chain, and CD3 molecules together constitute the “TCR complex”.

As used herein, the term “chimeric antigen receptor” or “CAR” has its general meaning in the art and refers to an artificially constructed hybrid protein or polypeptide containing the antigen binding domains of an antibody (e.g., scFv) linked to T-cell signalling domains. Characteristics of CARs include their ability to redirect T-cell specificity and reactivity toward a selected target in a non-MHC-restricted manner, exploiting the antigen-binding properties of monoclonal antibodies. Moreover, when expressed in T-cells, CARs advantageously do not dimerize with endogenous T cell receptor (TCR) alpha and beta chains. The chimeric antigen receptor the present invention typically comprises an extracellular hinge domain, a transmembrane domain, and an intracellular T cell signalling domain.

As used herein the term “CAR-T cell” refers to a T lymphocyte that has been genetically engineered to express a CAR. The T lymphocytes that are genetically modified may be “derived” or “obtained” from the patient who will receive the treatment using the genetically modified T cells or they may “derived” or “obtained” from a different patient. Thus the term “CAR-Treg” refers to a Treg that has been genetically engineered to express a CAR.

As used herein, the term “TCR-deficient CAR-Treg” refers to a CAR-Treg that lacks expression of a functional TCR complex.

As used herein, the term “CRISPR-associated endonuclease” has its general meaning in the art and refers to clustered regularly interspaced short palindromic repeats associated which are the segments of prokaryotic DNA containing short repetitions of base sequences. In bacteria the CRISPR/Cas loci encode RNA-guided adaptive immune systems against mobile genetic elements (viruses, transposable elements and conjugative plasmids). Three types (I-VI) of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA). The CRISPR-associated endonucleases Cas9 and Cpf1 belong to the type II and type V CRISPR/Cas system and have strong endonuclease activity to cut target DNA. Cas9 is guided by a mature crRNA that contains about 20 nucleotides of unique target sequence (called spacer) and a trans-activated small RNA (tracrRNA) that serves as a guide for ribonuclease Ill-aided processing of pre-crRNA. The crRNA: tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3^{rd} or the 4^{th} nucleotide from PAM). The crRNA and tracrRNA can be expressed separately or engineered into an artificial fusion small guide RNA (sgRNA) via a synthetic stem loop to mimic the natural crRNA/tracrRNA duplex. Such sgRNA, like shRNA, can be synthesized or in vitro transcribed for direct RNA transfection or expressed from U6 or H1-promoted RNA expression vector.

As used herein, the term “antigen” has its general meaning in the art and generally refers to a substance or fragment thereof that is recognized and selectively bound by an antibody or by a T cell antigen receptor, resulting in induction of an immune response. Antigens according to the invention are typically, although not exclusively, peptides and proteins. Antigens may be natural or synthetic and generally induce an immune response that is specific for that antigen.

As used herein, the term “auto-antigen” means any self-antigen arising from the own body tissues which is mistakenly recognized by the immune system as being foreign. Auto-antigens comprise, but are not limited to, cellular proteins, phosphoproteins, cellular surface proteins, cellular lipids, nucleic acids, glycoproteins, including cell surface receptors.

As used herein, the term “HLA-A2” has its general meaning in the art and refers to a HLA serotype within the HLA-A ‘A’ serotype group and is encoded by the HLA-A*02 allele group including the HLA-A*02:01, HLA-A*02:02, HLA-A*02:03, HLA-A*02:05, HLA-A*02:06, HLA-A*02:07 and HLA-A*02:11 gene products. HLA-A2 is very common in the Caucasian population (40-50%) and provides an ideal cellular target for the first portion because it will be suitable for use in a high proportion of combinations of HLA-A2+ donors and HLA-A2-recipients.

As used herein the term “antibody” and “immunoglobulin” have the same meaning, and will be used equally in the present invention. The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies and antibody fragments. In natural antibodies, two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes three (α, δ, γ) to five (μ, ε) domains, a variable domain (VH) and three to four constant domains (CH1, CH2, CH3 and CH4 collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain.

The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site. CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDR set from each of a heavy and a light chain V region. Framework Regions (FRs) refer to amino acid sequences interposed between CDRs. The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35B (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system.

As used herein, the terms “monoclonal antibody”, “monoclonal Ab”, “monoclonal antibody composition”, “mAb”, or the like, as used herein refer to a preparation of antibody molecules of single molecular composition. A monoclonal antibody is obtained from a population of substantially homogeneous antibodies, i.e., the individual antibodies comprised in the population are identical except for possible naturally occurring mutations that may be present in minor amounts.

As used herein the term “human antibody” as used herein, is intended to include antibodies having variable and constant regions derived from human immunoglobulin sequences. The human antibodies of the present invention may include amino acid residues not encoded by human immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

As used herein, the term “chimeric antibody” refers to an antibody which comprises a VH domain and a VL domain of a non-human antibody, and a CH domain and a CL domain of a human antibody. In some embodiments, a “chimeric antibody” is an antibody molecule in which (a) the constant region (i.e., the heavy and/or light chain), or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. Chimeric antibodies also include primatized and in particular humanized antibodies. Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992). (see U.S. Pat. No. 4,816,567; and Morrison et al., Proc. Natl. Acad. Sci. USA, 81:6851-6855 (1984)).

As used hereon, the term “humanized antibody” refers to an antibody having variable region framework and constant regions from a human antibody but retains the CDRs of a previous non-human antibody. In some embodiments, a humanized antibody contains minimal sequence derived from non-human immunoglobulin. For the most part, humanized antibodies and antibody fragments thereof may be human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. Such antibodies are designed to maintain the binding specificity of the non-human antibody from which the binding regions are derived, but to avoid an immune reaction against the non-human antibody. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321:522-525, 1986; Reichmann et al., Nature, 332:323-329, 1988; Presta, Curr. Op. Struct. Biol., 2:593-596, 1992.

As used herein, the term “antibody fragment” refers to at least one portion of an intact antibody, preferably the antigen binding region or variable region of the intact antibody, that retains the ability to specifically interact with (e.g., by binding, steric hindrance, stabilizing/destabilizing, spatial distribution) an epitope of an antigen. “Fragments” comprise a portion of the intact antibody, generally the antigen binding site or variable region. Examples of antibody fragments include Fab, Fab′, Fab′-SH, F(ab′) 2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific antibodies formed from antibody fragments. Fragments of the present antibodies can be obtained using standard methods.

As used herein, the term “scFv” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

As used herein, the term “Fc region” has its general meaning in the art and includes the polypeptides comprising the constant region of an antibody excluding the first constant region immunoglobulin domain. Thus, Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cgamma2 and Cgamma3 (Cγ2 and Cγ3) and the hinge between Cgamma1 (Cγ1) and Cgamma2 (Cγ2). Although the boundaries of the Fc region may vary, the human IgG heavy chain Fc region is usually defined to comprise residues C226 or P230 to its carboxyl-terminus, wherein the numbering is according to the EU index as in Kabat et al. (1991, NIH Publication 91-3242, National Technical Information Service, Springfield, Va.). The “EU index as set forth in Kabat” refers to the residue numbering of the human IgG1 EU antibody as described in Kabat et al. supra. Fc may refer to this region in isolation, or this region in the context of an antibody, antibody fragment, or Fc fusion protein. An Fc variant protein may be an antibody, Fc fusion, or any protein or protein domain that comprises an Fc region. Particularly preferred are proteins comprising variant Fc regions, which are non-naturally occurring variants of an Fc region. The amino acid sequence of a non-naturally occurring Fc region (also referred to herein as a “variant Fc region”) comprises a substitution, insertion and/or deletion of at least one amino acid residue compared to the wild type amino acid sequence. Any new amino acid residue appearing in the sequence of a variant Fc region as a result of an insertion or substitution may be referred to as a non-naturally occurring amino acid residue. Note: Polymorphisms have been observed at a number of Fc positions, including but not limited to Kabat 270, 272, 312, 315, 356, and 358, and thus slight differences between the presented sequence and sequences in the prior art may exist.

As used herein, the terms “Fc receptor” or “FcR” are used to describe a receptor that binds to the Fc region of an antibody. The primary cells for mediating ADCC express FcγRIII, whereas monocytes express FcγRI, FcγRII, FcγRIII and/or FcγRIV. FcR expression on hematopoietic cells is summarized in Ravetch and Kinet, Annu. Rev. Immunol., 9:457-92 (1991). To assess ADCC activity of a molecule, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecules of interest may be assessed in vivo, e.g., in an animal model such as that disclosed in Clynes et al., Proc. Natl. Acad. Sci. (USA), 95:652-656 (1998). As used herein, the term “effector cells” are leukocytes which express one or more FcRs and perform effector functions. The cells express at least FcγRI, FCγRII, FcγRIII and/or FcγRIV and carry out ADCC effector function. Examples of human leukocytes which mediate ADCC include peripheral blood mononuclear cells (PBMC), natural killer (NK) cells, monocytes, cytotoxic T cells and neutrophils.

As used herein, the term “anti-TCR complex antibody” refers to an antibody having binding specificity for one component of the TCR complex. According to the present invention the anti-TCR complex antibody of the present invention induces T cell hyporesponsiveness in the anergic state. In particular, the anti-TCR antibody of the present invention can modulate the TCR, induce apoptosis, and induce generalized long-term T cell unresponsiveness. The anti-TCR antibody can also induce internalization of the TCR complex and depletion of T cells from the circulation and peripheral lymphoid organs. In particular, the anti-TCR complex antibody triggers changes in the T cell receptor (TCR) complex, including (chain tyrosine phosphorylation and ZAP-70 association. In some aspects, the anti-TCR complex antibody of the present invention reduces the surface expression of the TCR complex on the T cell, and does not deplete T cells. Preferably, the anti-TCR complex antibody of the present invention is a “non-mitogenic antibody”, i.e. has properties to bind to the TCR complex antigen but without inducing production of a cytokine such as Tumor Necrosis Factor-alpha (TNF-α), Interferon-gamma (IFN-γ), Interleukin-2 and Interleukin-6. Thus, a non-mitogenic anti-TCR complex antibody delivers for instance a partial T cell signal that renders activated T cells unresponsive. In particular, the anti-TCR complex antibody is an anti-CD3 antibody.

As used herein, the term “anti-CD3 antibody” include antibodies and antigen-binding fragments thereof that specifically recognize a single CD3 subunit (e.g., epsilon, delta, gamma or zeta), as well as antibodies and antigen-binding fragments thereof that specifically recognize a dimeric complex of two CD3 subunits (e.g., gamma/epsilon, delta/epsilon, and zeta/zeta CD3 dimers). More particularly, the anti-CD3 antibody of the present invention binds to the epsilon (¿) chain of the CD3/TCR complex present at the surface of all peripheral T cells.

As used herein, the term “specificity” refers to the ability of an antibody to detectably bind target molecule (e.g. an epitope presented on an antigen) while having relatively little detectable reactivity with other target molecules. Specificity can be relatively determined by binding or competitive binding assays, using, e.g., Biacore instruments, as described elsewhere herein. Specificity can be exhibited by, e.g., an about 10:1, about 20:1, about 50:1, about 100:1, 10.000:1 or greater ratio of affinity/avidity in binding to the specific antigen versus nonspecific binding to other irrelevant molecules.

The term “affinity”, as used herein, means the strength of the binding of an antibody to a target molecule (e.g. an epitope). The affinity of a binding protein is given by the dissociation constant Kd. For an antibody said Kd is defined as [Ab]×[Ag]/[Ab-Ag], where [Ab-Ag] is the molar concentration of the antibody-antigen complex, [Ab] is the molar concentration of the unbound antibody and [Ag] is the molar concentration of the unbound antigen. The affinity constant Ka is defined by 1/Kd. Preferred methods for determining the affinity of a binding protein can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of binding protein is the use of Biacore instruments.

The term “binding” as used herein refers to a direct association between two molecules, due to, for example, covalent, electrostatic, hydrophobic, and ionic and/or hydrogen-bond interactions, including interactions such as salt bridges and water bridges. In particular, as used herein, the term “binding” in the context of the binding of an antibody to a predetermined target molecule (e.g. an antigen or epitope) typically is a binding with an affinity corresponding to a K_{D} of about 10⁻⁷ M or less, such as about 10⁻⁸ M or less, such as about 10⁻⁹ M or less, about 10⁻¹⁰ M or less, or about 10⁻¹¹ M or even less.

As used herein, the term “epitope” refers to a specific arrangement of amino acids located on a protein or proteins to which an antibody binds. Epitopes often consist of a chemically active surface grouping of molecules such as amino acids or sugar side chains, and have specific three-dimensional structural characteristics as well as specific charge characteristics. Epitopes can be linear or conformational, i.e., involving two or more sequences of amino acids in various regions of the antigen that may not necessarily be contiguous.

As used herein, the term “depletion” with respect to cancer cells, refers to a measurable decrease in the number of CD38 expressing cancer cells in the patient. The reduction can be at least about 10%, e.g., at least about 20%, 30%, 40%, 50%, 60%, 70%, 80%, 90%, 95%, 96%, 97%, 98%, 99%, or more. In some embodiments, the term refers to a decrease in the number of CD38 cancer cells in the patient below detectable limits.

As used herein, the term “tolerance” refers to a failure to respond, or a reduced response, to an antigen (including auto-antigens, allergens and endogenously administered molecules). This may mean that a productive (immunogenic) response is not induced upon endogenous or exogenous exposure to said antigen. This response may be replaced, in part or completely, by a tolerogenic response, i.e. an active process that further limits immunogenic responses. Examples of tolerogenic responses include, but are not limited to, generation of T regulatory cells, elimination of effector (conventional) T cells by apoptosis or their neutralization by anergy, and skewing of T cells and other immune cells towards phenotypes favouring a state of tolerance, e.g. production of regulatory cytokines such as interleukin-10 and TGF-β and of other anti-inflammatory mediators and downregulated expression of co-stimulatory molecules. These immunological concepts are well known to the skilled in the art.

As used herein, the term “transplantation” and variations thereof refers to the insertion of a transplant (also called graft) into a recipient, whether the transplantation is syngeneic (where the donor and recipient are genetically identical), allogeneic (where the donor and recipient are of different genetic origins but of the same species), or xenogeneic (where the donor and recipient are from different species). Thus, in a typical scenario, the host is human and the graft is an isograft, derived from a human of the same or different genetic origins. In another scenario, the graft is derived from a species different from that into which it is transplanted, including animals from phylogenically widely separated species, for example, a baboon heart being transplanted into a human host. In some embodiments the donor of the transplant is a human. The donor of the transplant can be a living donor or a deceased donor, namely a cadaveric donor. In some embodiments, the transplant is an organ, a tissue or cells.

As used herein, the expression “preventing or reducing transplant rejection” is meant to encompass prevention or inhibition of immune transplant rejection, as well as delaying the onset or the progression of immune transplant rejection. The term is also meant to encompass prolonging survival of a transplant in a patient, or reversing failure of a transplant in a patient. Further, the term is meant to encompass ameliorating a symptom of an immune transplant rejection, including, for example, ameliorating an immunological complication associated with immune rejection, such as for example, interstitial fibrosis, chronic graft arteriosclerosis, or vasculitis.

As used herein, the term “transplant rejection” encompasses both acute and chronic transplant rejection. “Acute rejection” is the rejection by the immune system of a tissue transplant recipient when the transplanted tissue is immunologically foreign. Acute rejection is characterized by infiltration of the transplant tissue by immune cells of the recipient, which carry out their effector function and destroy the transplant tissue. The onset of acute rejection is rapid and generally occurs in humans within a few weeks after transplant surgery. Generally, acute rejection can be inhibited or suppressed with immunosuppressive drugs such as rapamycin, cyclosporin and the like. “Chronic rejection” generally occurs in humans within several months to years after engraftment, even in the presence of successful immunosuppression of acute rejection. Fibrosis is a common factor in chronic rejection of all types of organ transplants.

As used herein, the term “Graft-Versus-Host Disease” or “GVHD” refers to a common and serious complication wherein there is a reaction of donated immunologically competent lymphocytes against a transplant recipient's own tissues. GVHD is a possible complication of any transplant that uses or contains hematopoietic stem cells from either a related or an unrelated donor.

As used herein, the term “autoimmune disease” refers to the presence of an autoimmune response (an immune response directed against an auto- or self-antigen) in a subject. Autoimmune diseases include diseases caused by a breakdown of self-tolerance such that the adaptive immune system, in concert with cells of the innate immune system, responds to self-antigens and mediates cell and tissue damage. In some embodiments, autoimmune diseases are characterized as being a result of, at least in part, a humoral and/or cellular immune response.

As used herein, the term “allergy” generally refers to an inappropriate immune response characterized by inflammation and includes, without limitation, food allergies, respiratory allergies and other allergies causing or with the potential to cause a systemic response such as, by way of example, Quincke's oedema and anaphylaxis.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used the terms “combination” and “combination therapy” are interchangeable and refer to treatments comprising the administration of the population of TCR-deficient CAR-Tregs together with the amount of the anti-CD3 antibody, separately or sequentially. As used herein the term “co-administering” as used herein means a process whereby the combination of the population of TCR-deficient CAR-Tregs together with the amount of the anti-CD3 antibody is administered to the same patient. The population of TCR-deficient CAR-Tregs together and the amount of the anti-CD3 antibody may be administered simultaneously, at essentially the same time, or sequentially. The population of TCR-deficient CAR-Tregs together and the amount of the anti-CD3 antibody can be administered separately by means of different vehicles or composition. The population of TCR-deficient CAR-Tregs together and the amount of the anti-CD3 antibody can also administered in the same vehicle or composition (e.g. pharmaceutical composition). The population of TCR-deficient CAR-Tregs together and the amount of the anti-CD3 antibody may be administered one or more times and the number of administrations of each component of the combination may be the same or different.

As used herein, the term “therapeutically effective combination” refers to an amount the population of TCR-deficient CAR-Tregs together with the amount of the anti-CD3 antibody that is sufficient to induce therapeutic effects (e.g. tolerance). The “therapeutically effective amount” is determined using procedures routinely employed by those of skill in the art such that an “improved therapeutic outcome” results. It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific polypeptide employed; and like factors well known in the medical arts. For example, it is well within the skill of the art to start doses of the compound at levels lower than those required to achieve the desired therapeutic effect and to gradually increase the dosage until the desired effect is achieved.

As used herein, the term “pharmaceutical composition” refers to a composition described herein, or pharmaceutically acceptable salts thereof, with other agents such as carriers and/or excipients. The pharmaceutical compositions as provided herewith typically include a pharmaceutically acceptable carrier.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical-Sciences, Sixteenth Edition, E. W. Martin (Mack Publishing Co., Easton, Pa., 1980) discloses various carriers used in formulating pharmaceutical compositions and known techniques for the preparation thereof.

### Methods of the Present Invention

The first object of the present invention relates to a method of inducing tolerance to one antigen of interest in a subject in need thereof comprising administering to the subject a therapeutically effective combination of a population of TCR-deficient CAR-Tregs cells specific for said antigen with an amount of an anti-TCR complex antibody.

### Diseases:

In some embodiments, the subject is predisposed or believed to be predisposed to developing, or has already developed or is developing, at least one symptom of a disease or condition caused by inappropriate or unwanted immune system activity against an antigen. The subject may be identified or diagnosed as having done so or as likely to do so based on a variety of factors, for example, family history and/or genetic testing of e.g. the mother and/or father, siblings, other relatives (grandparents, aunts, uncles, cousins, etc.), presence of other disease biomarkers such as (auto) antibodies directed against different (self-) antigens. Generally, the subject is known to have a genetic predisposition to development of an autoimmune disease, an allergy or other unwanted immune response. By “is known to have a genetic predisposition”, we mean that one or both parents or siblings may have the disease or condition, and/or are known to be carriers of gene(s) that is/are associated with the disease or condition, so that the statistically probability of the subject having or developing the disease is at least 1, 5, 10, 20, 30, 40, 50, 60, 70, 80, 90%, or is 100%. The determination may be based on observation of the health of the parents, siblings or of the subject, or on genetic testing of the same and identification of a gene or genes in a form known to be associated with or to cause the disease or condition, e.g. to have a particular sequence such as an allele, mutation, insertion, deletion, etc. The risk of disease may or may not also be confirmed by genotypying subject's cells and/or by assessing them by suitable biomarkers, including non-genetic biomarkers such as, by way of example, antibodies and other immune phenotypes or epigenetic modifications. Those of skill in the art will also recognize that such genetic traits may not be “all or nothing”, in that gene dosage may apply. Nevertheless, if a subject is deemed to be at risk, and if the life of a subject can be lengthened or improved by the practice of the present methods, then the subject is a viable candidate for treatment.

In some embodiments, the subject is predisposed or believed to be predisposed to developing, or has already developed or is developing an autoimmune disease. Examples of autoimmune disease include, without limitation, acute disseminated encephalomyelitis (ADEM), acute necrotizing hemorrhagic leukoencephalitis, Addison's disease, agammaglobulinemia, alopecia areata, amyloidosis, ankylosing spondylitis, anti-GBM/Anti-TBM nephritis, antiphospholipid syndrome (APS), autoimmune angioedema, autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune pancreatitis, autoimmune retinopathy, autoimmune thrombocytopenia purpura (ATP), autoimmune thyroid disease, autoimmune urticaria, axonal and neuronal neuropathies, Behcet's disease, bullous pemphigoid, autoimmune cardiomyopathy, Castleman disease, celiac disease, Chagas disease, chronic fatigue syndrome, chronic inflammatory demyelinating polyneuropathy (CIDP), chronic recurrent multifocal ostomyelitis (CRMO), Churg-Strauss syndrome, cicatricial pemphigoid/benign mucosal pemphigoid, Crohn's disease, Cogans syndrome, cold agglutinin disease, congenital heart block, coxsackie myocarditis, CREST disease, essential mixed cryoglobulinemia, demyelinating neuropathies, dermatitis herpetiformis, dermatomyositis, Devic's disease (neuromyelitis optica), discoid lupus, Dressler's syndrome, endometriosis, eosinophilic fasciitis, erythema nodosum, experimental allergic encephalomyelitis, Evans syndrome, fibromyalgia, fibrosing alveolitis, giant cell arteritis (temporal arteritis), glomerulonephritis, Goodpasture's syndrome, granulomatosis with polyangiitis (GPA), Graves' disease, Guillain-Barre syndrome, Hashimoto's encephalitis, Hashimoto's thyroiditis, hemolytic anemia, Henoch-Schonlein purpura, herpes gestationis, hypogammaglobulinemia, hypergammaglobulinemia, idiopathic thrombocytopenia purpura (ITP), IgA nephropathy, IgG4-related sclerosing disease, immunoregulatory lipoproteins, inclusion body myositis, inflammatory bowel disease, insulin-dependent diabetes (type 1), interstitial cystitis, juvenile arthritis, Kawasaki syndrome, Lambert-Eaton syndrome, leukocytoclastic vasculitis, lichen planus, lichen sclerosus, ligneous conjunctivitis, linear IgA disease (LAD), lupus (SLE), Lyme disease, Meniere's disease, microscopic polyangiitis, mixed connective tissue disease (MCTD), monoclonal gammopathy of undetermined significance (MGUS), Mooren's ulcer, Mucha-Habermann disease, multiple sclerosis, myasthenia gravis, myositis, narcolepsy, neuromyelitis optica (Devic's), autoimmune neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, paroxysmal nocturnal hemoglobinuria (PNH), Parry Romberg syndrome, Parsonnage-Turner syndrome, pars planitis (peripheral uveitis), pemphigus, peripheral neuropathy, perivenous encephalomyelitis, pernicious anemia, POEMS syndrome, polyarteritis nodosa, type I, II, & III autoimmune polyglandular syndromes, polymyalgia rheumatica, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasia, Raynaud's phenomenon, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm & testicular autoimmunity, stiff person syndrome, subacute bacterial endocarditis (SBE), Susac's syndrome, sympathetic ophthalmia, Takayasu's arteritis, temporal arteritis/Giant cell arteritis, thrombocytopenia purpura (TTP), Tolosa-Hunt syndrome, transverse myelitis, ulcerative colitis, undifferentiated connective tissue disease (UCTD), uveitis, vasculitis, vesiculobullous dermatosis, vitiligo, Waldenstrom's macroglobulinemia (WM), and Wegener's granulomatosis [Granulomatosis with Polyangiitis (GPA)].

In some embodiments, the subject is predisposed or believed to be predisposed to developing, or who has already developed or is developing an allergy. The term encompasses allergy, allergic disease, hypersensitive associated disease or respiratory disease associated with airway inflammation, such as asthma or allergic rhinitis. In some embodiments, the method of the present invention is effective in preventing, treating or alleviating one or more symptoms related to anaphylaxis, drug hypersensitivity, skin allergy, eczema, allergic rhinitis, urticaria, atopic dermatitis, dry eye disease, allergic contact allergy, food hypersensitivity, allergic conjunctivitis, insect venom allergy, bronchial asthma, allergic asthma, intrinsic asthma, occupational asthma, atopic asthma, acute respiratory distress syndrome (ARDS) and chronic obstructive pulmonary disease (COPD). Hypersensitivity associated diseases or disorders that may be treated by the method of the present invention include, but are not limited to, anaphylaxis, drug reactions, skin allergy, eczema, allergic rhinitis, urticaria, atopic dermatitis, dry eye disease [or otherwise referred to as Keratoconjunctivitis sicca (KCS), also called keratitis sicca, xerophthalmia], allergic contact allergy, food allergy, allergic conjunctivitis, insect venom allergy and respiratory diseases associated with airway inflammation, for example, IgE mediated asthma and non-IgE mediated asthma. The respiratory diseases associated with airway inflammation may include, but are not limited to, rhinitis, allergic rhinitis, bronchial asthma, allergic (extrinsic) asthma, non-allergic (intrinsic) asthma, occupational asthma, atopic asthma, exercise induced asthma, cough-induced asthma, acute respiratory distress syndrome (ARDS) and chronic obstructive pulmonary disease (COPD).

In some embodiments, the subject is predisposed or believed to be predisposed to developing, or has already developed or is developing an immune reaction against molecules that are exogenously administered for therapeutic or other purposes and may trigger an unwanted immune response. Non-limiting examples of this kind include immune reactions against replacement therapeutics in the context of genetic deficiencies, which include, but are not limited to, haemophilia A, haemophilia B, congenital deficiency of other clotting factors such as factor II, prothrombin and fibrinogen, primary immunodeficiencies (e.g. severe combined immunodeficiency, X-linked agammaglobulinemia, IgA deficiency), primary hormone deficiencies such as growth hormone deficiency and leptin deficiency, congenital enzymopathies and metabolic disorders such as disorders of carbohydrate metabolism (e.g. sucrose-isomaltase deficiency, glycogen storage diseases), disorders of amino acid metabolism (e.g. phenylketonuria, maple syrup urine disease, glutaric acidemia type 1), urea cycle disorders (e.g. carbamoyl phosphate synthetase I deficiency), disorders of organic acid metabolism (e.g. alcaptonuria, 2-hydroxyglutaric acidurias), disorders of fatty acid oxidation and mitochondrial metabolism (e.g. medium-chain acyl-coenzyme A dehydrogenase deficiency), disorders of porphyrin metabolism (e.g. porphyrias), disorders of purine or pyrimidine metabolism (e.g. Lesch-Nyhan syndrome), disorders of steroid metabolism (e.g. lipoid congenital adrenal hyperplasia, congenital adrenal hyperplasia), disorders of mitochondrial function (e.g. Kearns-Sayre syndrome), disorders of peroxisomal function (e.g. Zellweger syndrome), lysosomal storage disorders (e.g. Gaucher's disease, Niemann Pick disease). In the case of genetic deficiencies, the proposed method may not only allow to reinstate immune tolerance against the replacement therapeutics that are used to treat the disease, but also reinstate the biological activity for which said therapeutics are administered. Other therapeutics for which said method may be suitable to limit undesired immune responses include other biological agents such as, by way of example, cytokines, monoclonal antibodies, receptor antagonists, soluble receptors, hormones or hormone analogues, coagulation factors, enzymes, bacterial or viral proteins. For example, hemophilic children can be treated prophylactically with periodic coagulation factor (e.g. factor VIII) replacement therapy, which decreases the chance of a fatal bleed due to injury. In addition to the expense and inconvenience of such treatment, repeated administration results in inhibitor antibody formation in some patients against the coagulation factor. If the antibodies in these patients are low titer antibodies, patients are treated with larger doses of blood coagulation factors. If the antibodies are high titer antibodies, treatment regimens for these patients become much more complex and expensive. In some embodiments, the therapeutic protein is produced in the subject following gene therapy suitable e.g. for the treatment of congenital deficiencies. Gene therapy typically involves the genetic manipulation of genes responsible for disease. One possible approach for patients, like those with hemophilia deficient for a single functional protein, is the transmission of genetic material encoding the protein of therapeutic interest. However, the repeated administration of gene therapy vectors, such as viral vectors, may also trigger unwanted immune responses against the therapeutic protein introduced in the vector and/or against other components of the vector. Thus, the method of the present invention can be suitable for overcoming the body's immune response to gene therapy vectors such as viral vectors. Viral vectors are indeed the most likely to induce an immune response, especially those, like adenovirus and adeno-associated virus (AAV), which express immunogenic epitopes within the organism. Various viral vectors are used for gene therapy, including, but not limited to, retroviruses for X-linked severe combined immunodeficiency (X-SCID); adenoviruses for various cancers; adeno-associated viruses (AAVs) to treat muscle and eye diseases; lentivirus, herpes simplex virus and other suitable means known in the art.

In some embodiments, the subject is predisposed or believed to be predisposed to developing, or has already developed or is developing an immune reaction against a grafted organ, a grafted tissue, or a grafted population of cells. Typically the subject may have been transplanted with a graft selected from the group consisting of heart, kidney, lung, liver, pancreas, pancreatic islets, brain tissue, stomach, large intestine, small intestine, cornea, skin, trachea, bone, bone marrow, muscle, or bladder. The method of the invention is indeed particularly suitable for preventing or suppressing an immune response associated with rejection of a donor tissue, cell, graft, or organ transplant by a recipient subject. Graft-related diseases or disorders include graft versus host disease (GVDH), such as associated with bone marrow transplantation, and immune disorders resulting from or associated with rejection of organ, tissue, or cell graft transplantation (e.g., tissue or cell allografts or xenografts), including, e.g., grafts of skin, muscle, neurons, islets, organs, parenchymal cells of the liver, etc. With regard to a donor tissue, cell, graft or solid organ transplant in a recipient subject, it is believed that the adoptive immunotherapy according to the invention may be effective in preventing acute rejection of such transplant in the recipient and/or for long-term maintenance therapy to prevent rejection of such transplant in the recipient (e.g., inhibiting rejection of insulin-producing islet cell transplant from a donor in the subject recipient suffering from diabetes). Thus the adoptive immunotherapy of the invention is useful for preventing Host-Versus-Graft-Disease (HVGD) and Graft-Versus-Host-Disease (GVHD). Typically, the TCR-deficient CAR-Treg cells may be administered to the subject before and/or after transplantation. In some embodiments, the TCR-deficient CAR-Treg cells may be administered to the subject on a periodic basis before and/or after transplantation.

### Antigens:

In some embodiments, the antigen is an auto-antigen. Auto-antigens comprise, but are not limited to, cellular proteins, phosphoproteins, cellular surface proteins, cellular lipids, nucleic acids, glycoproteins, including cell surface receptors. It may be, by way of example, an auto-antigen of the following non-limiting list: acethylcholine receptor, actin, adenin nucleotide translocator, β-adrenoreceptor, aromatic L-amino acid decarboxylase, asioaloglycoprotein receptor, bactericidal/permeability increasing protein (BPi), calcium sensing receptor, cholesterol side chain cleavage enzyme, collagen type IV αγ-chain, cytochrome P450 2D6, desmin, desmoglein-1, desmoglein-3, F-actin, GM-gangliosides, glutamate decarboxylase, glutamate receptor, H/K ATPase, 17-α-hydroxylase, 21-hydroxylase, IA-2 (ICAS12), insulin, insulin receptor, intrinsic factor type 1, leucocyte function antigen 1, myelin associated glycoprotein, myelin basic protein, myelin oligodendrocyte protein, myosin, P80-coilin, pyruvate deshydrogenase complex E2 (PDC-E2), sodium iodide symporter, SOX-10, thyroid and eye muscle shared protein, thyroglobulin, thyroid peroxydase, thyrotropin receptor, tissue transglutaminase, transcription coactivator p75, tryptophan hydroxylase, tyrosinase, tyrosine hydroxylase, ACTH, aminoacyl-tRNA-hystidyl synthetase, cardiolipin, carbonic anhydrase II, cebtromere associated proteins, DNA-dependant nucleosome-stimulated ATPase, fibrillarin, fibronectin, glucose 6 phosphate isomerase, beta 2-glycoprotein I, golgin (95, 97, 160, 180), heat shock proteins, hemidesmosomal protein 180, histone H2A, H2B, keratin, IgE receptor, Ku-DNA protein kinase, Ku-nucleoprotein, La phosphoprotein, myeloperoxydase, proteinase 3, RNA polymerase I-III, signal recognition protein, topoisomerase I, tubulin, vimenscin, myelin associated oligodendrocyte basic protein (MOBP), proteolipid protein, oligodendrocyte specific protein (OSP/Claudin 11), cyclic nucleotide 3′ phosphodiesterase (CNPase), BP antigen 1 (BPAGI-e), transaldolase (TAL), human mitochondrial autoantigens PDC-E2 (Novo 1 and 2), OGDC-E2 (Novo 3), and BCOADC-E2 (Novo 4), bullous pemphigoid (BP) 180, laminin 5 (LN5), DEAD-box protein 48 (DDX48) or insulinoma-associated antigen-2.

In some embodiments, when the subject suffers from multiple sclerosis, the autoantigen is selected from the group consisting of myelin-related antigens (e.g. myelin basic protein (MBP) (e.g. MBP83-102 peptide), myelin oligodendrocyte glycoprotein (MOG) (e.g. MOG35-55 peptide) and proteolipid protein (PLP) (e.g. PLP139-151 peptide). When the subject suffers from Type I diabetes (TID), the autoantigen is selected from the group consisting of insulin, insulin precursor proinsulin (Prolns), glutamic acid decarboxylase 65 (GAD65), glial fibrillary acidic protein (GFAP), islet-specific glucose-6-phosphatase catalytic subunit-related protein (IGRP), insulinoma-associated antigen-2 (IA-2) and zinc transporter 8 (ZnT8). When the autoimmune disease is rheumatoid arthritis, the autoantigen is type II collagen (CTII).

In some embodiments, the antigen is a food antigen. In some embodiments, the food antigen is selected from non-limiting list: bovine antigens such as lipocalin, Ca-binding S100, alpha-lactalbumin, lactoglobulins such as beta-lactoglobulin, bovine serum albumin, caseins. Food-antigens may also be atlantic salmon antigens such as parvalbumin, chicken antigens such as ovomucoid, ovalbumin, Ag22, conalbumin, lysozyme or chicken serum albumin, peanuts, shrimp antigens such as tropomyosin, wheat antigens such as agglutinin or gliadin, celery antigens such as celery profilin, carrot antigens such as carrot profilin, apple antigens such as thaumatin, apple lipid transfer protein, apple profilin, pear antigens such as pear profilin, isoflavone reductase, avocado antigens such as endochitinase, apricot antigens such as apricot lipid transfer protein, peach antigens such as peach lipid transfer protein or peach profilin, soybean antigens such as HPS, soybean profilin or (SAM22) PR-10 prot.

In some embodiments, the antigen is an inflammatory antigen selected from the group consisting of to myelin basic protein (MBP), myelin associated glycoprotein (MAG), myelin oligodendrocyte glycoprotein (MOG), proteolipid protein (PLP), oligodendrocyte myelin oligoprotein (OMGP), myelin associated oligodendrocyte basic protein (MOBP), oligodendrocyte specific protein (OSP/Claudin 1), heat shock proteins, oligodendrocyte specific proteins (OSP), NOGO A, glycoprotein Po, peripheral myelin protein 22 (PMP22), 2′,3′-cyclic nucleotide 3-phosphodiesterase (CNPase), citrulline-substituted cyclic and linear filaggrin peptides, type II collagen peptides, human cartilage glycoprotein 39 (HCgp39) peptides, HSP, heterogeneous nuclear ribonucleoprotein (hnRNP) A2 peptides, hnRNP BI, hnRNP D, Ro60/52, HSP60, 65, 70 and 90, BiP, keratin, vimentin, fibrinogen, collagen type I, III, IV and V peptides, annexin V, Glucose 6 phosphate isomerase (GPI), acetyl-calpastatin, pyruvate deshydrogenase (PDH), aldolase, topoisomerase I, snRNP, PARP, Scl-70, Scl-100, phospholipid antigen including anionic cardiolipin and phosphatidylserine, neutrally charged phosphatidylethanolamine and phosphatidylcholine, matrix metalloproteinase, fibrillin, and aggrecan.

In some embodiments, the antigen is an allergen in particular an inhaled allergen, an ingested allergen or a contact allergen. Examples of allergens include, but are not limited to, inhaled allergens derived from pollens (Cup, Jun), house dust mites (Der, Gly, Tyr, Lep), dog, cat and rodents (Can, Fel, Mus, Rat). Examples of contact allergens include, but are not limited to, heavy metals (such as nickel, chrome, gold), latex, haptens such as halothane, hydralazine.

In some embodiments, the antigen is as an allo-antigen. Allo-antigens include, but are not limited to, antigens expressed by the allograft, proteins expressed in the course of gene therapy (and also viral antigens issued from the viral vector used) as well as therapeutic proteins. The alloantigen is selected from the group consisting of HLA-A, HLA-B, HLA-C, HLA-E, HLA-F, HLA-G, HLA-DM, HLA-DO, HLA-DP, HLA-DQ, HLA-DR, minor antigens such as HA-1, HA-2, HA-3, HA-8, HB-1, UGT2B 17, BCL2A1, HY B7, HY A2, HY A1, HY B60, HY B8, HY DQ5, HY DRB3, and blood group antigens A and B. In some embodiments, the alloantigen is HLA-A2.

In some embodiments, the antigen is a molecule that is exogenously administered for therapeutic or other purposes and may trigger an unwanted immune response. While frequently neutralising the biological activity that said molecules are meant to induce, such immune responses may have additional deleterious effects unrelated to the purpose for which the molecules were originally administered. Examples of this kind include immune reactions against therapeutic clotting factor VIII in haemophilia A or factor IX in haemophilia B, against different enzymes in congenital enzymopathies and, more in general, during any kind of replacement therapies in the context of genetic deficiencies. Allo-immunization responses against antigens expressed by tissues or hematopoietic and/or blood cells transplanted into an individual are equally considered.

### CAR-Treg Cells:

The TCR-deficient CAR-Treg cells of the present thus comprises a CAR having specificity for the antigen of interest.

According to the present invention, the CAR typically comprises an ectodomain (extracellular domain) and an endodomain (cytoplasmic domain), joined by a transmembrane domain. The ectodomain, expressed on the surface of the cell, comprises an antigen binding domain or receptor domain and optionally a spacer (or hinge) region linking the antigen binding domain to the transmembrane domain. The transmembrane domain is typically a hydrophobic alpha helix that spans across the lipid bilayer of the cell membrane. The endodomain of the CAR is composed of an intracellular signalling module that induces Treg cell activation upon antigen binding. The endodomain may include several signalling domains, as explained infra.

The extracellular domain of the CAR thus comprises an antigen binding domain that specifically binds or recognizes the antigen of interest.

In some embodiments, such antigen binding domain is an antibody, preferably a single chain antibody. Preferably, the antibody is a humanized antibody. Particularly, such antigen binding domain is an antibody fragment selected from fragment antigen binding (Fab) fragments, F(ab′) 2 fragments, Fab′ fragments, Fv fragments, recombinant IgG (rlgG) fragments, single chain antibody fragments, single chain variable fragments (scFv), single domain antibodies (e.g., sdAb, sdFv, nanobody) fragments, diabodies, and multi-specific antibodies formed from antibody fragments. In some embodiments, the antibodies are single-chain antibody fragments comprising a variable heavy chain region and/or a variable light chain region, such as scFv. Particularly, such antigen binding domain is selected from a Fab and a scFv.

In some embodiments, when the antigen targeting domain is a scFv, the scFv can be derived from the variable heavy chain (VH) and variable light chain (VL) regions of an antigen-specific mAb linked by a flexible linker. The scFv retains the same specificity and a similar affinity as the full antibody from which it is derived. The peptide linker connecting scFv VH and VL domains joins the carboxyl terminus of one variable region domain to the amino terminus of the other variable domain without compromising the fidelity of the VH-VL paring and antigen-binding sites. Peptide linkers can vary from 10 to 30 amino acids in length. In some embodiments, the scFv peptide linker is a Gly/Ser linker and comprises one or more repeats of the amino acid sequence Gly-Gly-Gly-Ser or Gly-Gly-Gly-Gly-Ser.

In some embodiments, the scFv is specific for HLA-A2. In some embodiments, the scFv comprises the amino acid sequence as set forth in SEQ ID NO:1.

SEQ ID NO: 1
>scFv_anti-HLA-A2
DVVMTQSPSSLSASVGDRVTITCQASQDISNYLNWYQQKPGKAPKLLIYD
ASNLETGVPSRFSGSGSGTDFTFTISSLQPEDFATYYCQQYSSFPLTFGG
GTKVDIKGGGGSGGGGSGGGGSQVQLVQSGGGVVQPGGSLRVSCAASGVT
LSDYGMHWVRQAPGKGLEWMAFIRNDGSDKYYADSVKGRFTISRDNSKKT
VSLQMSSLRAEDTAVYYCAKNGESGPLDYWYFDLWGRGTLVTV

The CAR optionally comprises a spacer or hinge domain linking the antigen binding domain to the transmembrane domain.

In some embodiments, the CAR comprises a hinge sequence between the antigen binding domain and the transmembrane domain and/or between the transmembrane domain and the cytoplasmic domain. One ordinarily skilled in the art will appreciate that a hinge sequence is a short sequence of amino acids that facilitates flexibility. In particular, the spacer or hinge domain linking the antigen binding domain to the transmembrane domain is designed to be sufficiently flexible to allow the antigen binding domain to orient in a manner that allows antigen recognition. The hinge may be derived from or include at least a portion of an immunoglobulin Fc region, for example, an IgG1 Fc region, an IgG2 Fc region, an IgG3 Fc region, an IgG4 Fc region, an IgE Fc region, an IgM Fc region, or an IgA Fc region. In certain embodiments, the hinge domain includes at least a portion of an IgG1, an IgG2, an IgG3, an IgG4, an IgE, an IgM, or an IgA immunoglobulin Fc region that falls within its CH2 and CH3 domains. Exemplary hinges include, but are not limited to, a CD8a hinge, a CD28 hinge, IgG1/IgG4 (hinge-Fc part) sequences, IgG4 hinge alone, IgG4 hinge linked to CH2 and CH3 domains, or IgG4 hinge linked to the CH3 domain, those described in Hudecek et al. (2013) Clin. Cancer Res., 19:3153, international patent application publication number WO2014031687, U.S. Pat. No. 8,822,647 or published app. No. US2014/0271635. As hinge domain, the invention relates to all or a part of residues 118 to 178 of CD8a (GenBank Accession No. NP_001759.3), residues 135 to 195 of CD8 (GenBank Accession No. AAA35664), residues 315 to 396 of CD4 (GenBank Accession No. NP_000607.1), or residues 137 to 152 of CD28 (GenBank Accession No. NP_006130.1) can be used. Also, as the spacer domain, a part of a constant region of an antibody H chain or L chain (CHI region or CL region) can be used. Further, the spacer domain may be an artificially synthesized sequence. In some embodiments, for example, the hinge sequence is derived from a CD8 alpha molecule or a CD28 molecule.

The transmembrane domain of the CAR functions to anchor the receptor on the cell surface. The choice of the transmembrane domain may depend on the neighbouring spacer and intracellular sequences.

In some embodiments, the transmembrane domain is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 zeta, CD3 epsilon, CD3 gamma, CD3 delta, CD45, CD4, CD5, CD8, CD9, CD16, CD22, CD33, CD37, CD64, CD80, CD86, CD134, CD137, CD154, ICOS/CD278, GITR/CD357, NKG2D, and DAP molecules. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. A transmembrane domain is thermodynamically stable in a membrane.

It may be a single alpha helix, a transmembrane beta barrel, a beta-helix of gramicidin A, or any other structure. Optionally, a short oligo- or polypeptide linker, preferably between 2 and 10 amino acids in length may form the linkage between the transmembrane domain and the intracellular signalling domain(s) of the CAR. A glycine-serine doublet may provide a suitable linker.

The role of the intracellular domain of the CAR is to produce an activation signal to the Treg cell as soon as the extracellular domain has recognized the antigen. In particular, the intracellular domain of the CAR triggers or elicits activation of at least one of the normal effector functions of the Treg cell. Examples of intracellular domain sequences that are of particular use in the invention include those derived from an intracellular signalling domain of a lymphocyte receptor chain, a TCR/CD3 complex protein, an Fc receptor subunit, an IL-2 receptor subunit, CD3ζ, FcRγ, FcRβ, CD3γ, CD3δ, CD3ε, CD5, CD22, CD79a, CD79b, CD66d, CD278 (ICOS), FcsRI, DAP10, and DAP12. It is particularly preferred that the intracellular domain in the CAR comprises a cytoplasmic signalling sequence derived from CD32. The intracellular domain of the CAR can be designed to comprise a signalling domain (such as the CD35 signalling domain) by itself or combined with costimulatory domain(s). A costimulatory molecule can be defined as a cell surface molecule that is required for an efficient response of lymphocytes to an antigen. Examples of such molecules include CD27, CD28, 4-1BB (CD137), OX40 (CD134), CD30, CD40, CD244 (2B4), ICOS, lymphocyte function-associated antigen-1 (LFA-1), CD2, CD7, LIGHT, NKG2C, B7-H3, and a ligand that specifically binds with CD83, CD8, CD4, b2c, CD80, CD86, DAP10, DAP12, MyD88, BTNL3, and NKG2D. The intracellular signalling portion of the above recited co-stimulatory domains can be used alone or in combination with other co-stimulatory domains. In particular, the CAR can comprise any combination of two or more co-stimulatory domains from the group consisting of CD27, CD28, 4-1BB (CD137), OX40 (CD134), CD30, CD40, CD244 (2B4), ICOS, LFA-1, CD2, CD7, LIGHT, NKG2C, B7-H3, and a ligand that specifically binds with CD83, CD8, CD4, b2c, CD80, CD86, DAP10, DAP12, MyD88, BTNL3, and NKG2D.

The CAR of the invention may be a first generation, a second generation, or a third generation CAR as described hereabove. Preferably, the CAR is a second or third generation CAR. Typically, “first-generation CARs” contain a single signalling domain. CARs containing a signalling domain together with one additional costimulatory domain are termed “second generation” while those containing a signalling domain together with two additional costimulatory domains are listed as “third generation”. For example, first-generation CARs contain solely the CD3ζ chain as a single signalling domain. Second- and third-generation CARs consist of one or two additional costimulatory signalling domains, respectively, such as CD28, CD27, OX-40 (CD134) and 4-1BB (CD137). For example, second-generation CAR may contain CD3ζ and CD28 signalling domains, while third-generation CAR may contain CD3ζ, CD28 and either OX40 (CD134) or 4-1BB (CD137). “TRUCKs” represent the recently developed “fourth-generation” CARs. TRUCKs (T cells redirected for universal cytokine killing) are CAR-redirected T cells used as vehicles to produce and release a transgenic product that accumulates in the targeted tissue. The product, for example a pro-inflammatory cytokine, may be constitutively produced or induced once the T cell is activated by the CAR. Other substances such as enzymes or immunomodulatory molecules may be produced in the same way and deposited by CAR-redirected T cells in the targeted lesion. This strategy involves two separate transgenes expressing for example (i) the CAR and (ii) a cell activation responsive promoter linked to a cytokine such as IL-12. Consequently, immune stimulatory cytokine such as IL-12 is secreted upon CAR engagement. In a particular embodiment, the CAR is a CAR of fourth generation as defined above.

Thus, for example, the CAR can be designed to comprise a signalling domain such as the CD35 signalling domain and a least one co-stimulatory signalling domain selected from CD28 and CD40, CD28 and 4-1BB (CD137), CD28 and OX40 (CD134), and CD28 and LFA-1. In some embodiments, the CAR of the present invention comprises the comprises the CD3ζ signalling domain and the 4-1BB co-stimulatory signalling domain as described in Lamarthée B, Marchal A, Charbonnier S, Blein T, Leon J, Martin E, Rabaux L, Vogt K, Titeux M, Delville M, Vinçon H, Six E, Pallet N, Michonneau D, Anglicheau D, Legendre C, Taupin J L, Nemazanyy I, Sawitzki B, Latour S, Cavazzana M, André I, Zuber J. Transient mTOR inhibition rescues 4-1BB CAR-Tregs from tonic signal-induced dysfunction. Nat Commun. 2021 Nov. 8;12 (1): 6446. doi: 10.1038/s41467-021-26844-1. PMID: 34750385; PMCID: PMC8575891.

In some embodiments, the CAR of the present invention is specific for HLA-A2. In some embodiments, the CAR comprises the amino acid sequence as set forth in SEQ ID NO:2.

SEQ ID NO: 2
>CAR (scFv anti-HLA2_signal-CD3z)
DVVMTQSPSSLSASVGDRVTITCQASQDISNYLNWYQQKPGKAPKLLIYD
ASNLETGVPSRFSGSGSGTDFTFTISSLQPEDFATYYCQQYSSFPLTFGG
GTKVDIKGGGGSGGGGSGGGGSQVQLVQSGGGVVQPGGSLRVSCAASGVT
LSDYGMHWVRQAPGKGLEWMAFIRNDGSDKYYADSVKGRFTISRDNSKKT
VSLQMSSLRAEDTAVYYCAKNGESGPLDYWYFDLWGRGTLVTVTTTPAPR
PPTPAPTIASQPLSLRPEACRPAAGGAVHTRGLDFACDFWVLVVVGGVLA
CYSLLVTVAFIIFWVRSKRSRLLHSDYMNMTPRRPGPTRKHYQPYAPPRD
FAAYRSRVKFSRSADAPAYKQGQNQLYNELNLGRREEYDVLDKRRGRDPE
MGGKPRRKNPQEGLYNELQKDKMAEAYSEIGMKGERRRGKGHDGLYQGLS
TATKDTYDALHMQALPPR

In a particular embodiment, multiple CARs such as CARs binding to different antigens, may be expressed by a single Treg-cell.

Methods for preparing CAR-T cells are well known in the art. Typically, the T cells are transduced in order to express one or more CAR. It is contemplated that a nucleic acid construct encoding the CAR can be introduced into the Treg cells as naked DNA or in a suitable vector. Naked DNA generally refers to the DNA encoding a chimeric receptor contained in a plasmid expression vector in proper orientation for expression. Physical methods for introducing a nucleic acid construct into a host cell include calcium phosphate precipitation, lipofection, particle bombardment, microinjection, electroporation, and the like. Other means can be used including colloidal dispersion systems, such as macromolecule complexes, nanocapsules, microspheres, beads, and lipid-based systems including oil-in-water emulsions, micelles, mixed micelles, and liposomes. In some embodiments, the nucleic acid construct encoding the CAR is introduced into the Treg cell by a viral vector such as retroviruses, adenoviruses, adeno-associated viruses, herpesviruses and lentiviruses. In particular, the vector is a lentiviral vector. On order to confirm the presence of the CAR sequence in the Treg cell, a variety of assays may be performed. Such assays include, for example, “molecular biological” assays well known such as Southern and Northern blotting, RT-PCR and quantitative PCR; “biochemical” assays, such as detecting the presence or absence of a particular peptide; “immunological” assays, such as detecting expression of the CAR at the cell surface by flow cytometry. In some embodiments, the antigen binding domain of a CAR of the invention (e.g., a scFv) is encoded by a nucleic acid molecule whose sequence has been codon optimized for expression in a mammalian cell. In some embodiments, entire CAR construct of the invention is encoded by a nucleic acid molecule whose entire sequence has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, e.g., methods disclosed in at least U.S. Pat. Nos. 5,786,464 and 6,114,148.

### TCR-Deficient CAR-Treg Cells:

Methods for preparing TCR-deficient CAR-T cells are well known in the art. This may e.g., be accomplished by different means as described infra, e.g., by engineering a T cell such that it does not express any functional TCR on its cell surface, or by engineering the T cell such that it does not express one or more of the subunits that comprise a functional TCR and therefore does not produce a functional TCR or by engineering a T cell such that it produces very little functional TCR on its surface, or which expresses a substantially impaired TCR, e.g. by engineering the T cell to express mutated or truncated forms of one or more of the subunits that comprise the TCR, thereby rendering the T cell incapable of expressing a functional TCR or resulting in a cell that expresses a substantially impaired TCR.

In some embodiments, the method includes introducing into a T cell a genome-editing nuclease designed to edit the TRAC coding region that encodes TCRa, and culturing the T cell under conditions for the genome-editing nuclease to modify the TRAC coding region to inhibit expression of TCRa. In some embodiments, introducing the genome-editing nuclease into the T cell includes introducing into the T cell a polynucleotide that encodes the genome-editing nuclease. In some embodiments, the genome-editing nuclease includes a TALEN nuclease, a CRISPR-associated endonuclease, or a megaTAL nuclease.

In some embodiments, the genome-editing nuclease is a CRISPR-associated endonuclease. CRISPR/Cas systems for gene editing in eukaryotic cells typically involve (1) a guide RNA molecule (gRNA) comprising a targeting sequence (which is capable of hybridizing to the genomic DNA target sequence), and sequence which is capable of encoding for the CRISPR-associated endonuclease.

In some embodiments, the CRISPR-associated endonuclease is a Cas9 nuclease. The Cas9 nuclease can have a nucleotide sequence identical to the wild type Streptococcus pyrogenes sequence. In some embodiments, the CRISPR-associated endonuclease can be a sequence from other species, for example other Streptococcus species, such as thermophilus; Pseudomona aeruginosa, Escherichia coli, or other sequenced bacteria genomes and archaea, or other prokaryotic microorganisms. Alternatively, the wild type Streptococcus pyogenes Cas9 sequence can be modified. The nucleic acid sequence can be codon optimized for efficient expression in mammalian cells, i.e., “humanized.” A humanized Cas9 nuclease sequence can be for example, the Cas9 nuclease sequence encoded by any of the expression vectors listed in Genbank accession numbers KM099231.1 GL669193757; KM099232.1 GL669193761; or KM099233.1 GL669193765. Alternatively, the Cas9 nuclease sequence can be for example, the sequence contained within a commercially available vector such as pX330, pX260 or pMJ920 from Addgene (Cambridge, MA). In some embodiments, the Cas9 endonuclease can have an amino acid sequence that is a variant or a fragment of any of the Cas9 endonuclease sequences of Genbank accession numbers KM099231.1 GL669193757; KM099232.1; GL669193761; or KM099233.1 GL669193765 or Cas9 amino acid sequence of pX330, pX260 or pMJ920 (Addgene, Cambridge, MA). Artificial CRISPR/Cas systems can be generated, using technology known in the art, e.g., that are described in U.S. Publication No. 20140068797, WO2015/048577, and Cong (2013) Science 339:819-823. Other artificial CRISPR/Cas systems that are known in the art may also be generated, e.g., that described in Tsai (2014) Nature Biotechnol., 32:6 569-576, U.S. Pat. Nos. 8,871,445; 8,865,406; 8,795,965; 8,771,945; and 8,697,359, the contents of which are hereby incorporated by reference in their entirety. Such systems can be generated by, for example, engineering a CRISPR/Cas system to include a gRNA molecule comprising a targeting sequence that hybridizes to a sequence of the TRAC gene. In some embodiments, the gRNA comprises a targeting sequence which is fully complementarity to 15-25 nucleotides, e.g., 20 nucleotides, of the TRAC gene. In some embodiments, the 15-25 nucleotides, e.g., 20 nucleotides, of the TRAC gene, are disposed immediately 5′ to a protospacer adjacent motif (PAM) sequence recognized by the Cas protein of the CRISPR/Cas system (e.g., where the system comprises a S. pyogenes Cas9 protein, the PAM sequence comprises NGG, where N can be any of A, T, G or C). In some embodiments, foreign DNA (e.g., DNA encoding a CAR) can be introduced into the cell along with the CRISPR/Cas system. In some embodiments, the contacting of the cells with the endonuclease system is done ex vivo. In embodiments, the contacting is done prior to, simultaneously with, or after said cells are modified to express a CAR, e.g., a CAR as described herein.

In some embodiments, the preparation of the TCR-deficient CAR-T cells is performed as described in the EXAMPLE.

In some embodiments, the expression of at least one immune checkpoint protein (e.g. PD-1, CTLA-4) is also repressed in the cells.

### Methods of Manufacturing TCR-Deficient CAR-Treg Cells:

Thus, a further object of the present invention relates to a method of manufacturing a TCR-deficient CAR-Treg cell of the present invention, comprising the steps consisting of i) introducing nucleic acid encoding a CAR into a cell and ii) contacting the cell with a endonuclease system so as to repress the expression of TCR complex.

In some embodiments, the method comprises the steps consisting of i) introducing nucleic acid encoding a CAR into a cell and ii) contacting the cell with a Cas protein and with at least one guide RNA molecules (gRNA) comprising a sequence that targets the TRAC gene, and a sequence which is capable of binding to the Cas protein.

In some embodiments, the cell is also contacted with at least one guide RNA molecule that comprising a sequence that targets a gene encoding for an immune checkpoint protein (e.g. PD-1, CTLA-4 . . . ).

Once the population of TCR-deficient CAR-Treg cells is obtained, functionality of the cells may be evaluated according to any standard method which typically include a cytotoxic assay. Cell surface phenotype of the cells with the appropriate binding partners can also be confirmed. Quantifying the secretion of various cytokines may also be performed. Methods for quantifying secretion of a cytokine in a sample are well known in the art. For example, any immunological method such as but not limited to ELISA, multiplex strategies, ELISPOT, immunochromatography techniques, proteomic methods, Western blotting, FACS, or Radioimmunoassays may be applicable to the present invention.

The population of CAR-Treg cells prepared as described above can be utilized in methods and compositions for adoptive immunotherapy in accordance with known techniques, or variations thereof that will be apparent to those skilled in the art based on the instant disclosure. See, e.g., US Patent Application Publication No. 2003/0170238 to Gruenberg et al; see also U.S. Pat. No. 4,690,915 to Rosenberg.

Currently, most adoptive immunotherapies are autolymphocyte therapies (ALT) directed to treatments using the patient's own immune cells. These therapies involve processing the patient's own lymphocytes to enhance the tolerance response towards specific antigens. Typically, the treatments are accomplished by removing the patient's lymphocytes and exposing these cells in vitro to biologics and drugs to convey them to a Treg profile. Once the Treg cells are prepared with the CAR of the present invention, these ex vivo cells are reinfused into the patient to enhance the immune system to induce tolerance. In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin. A treatment-effective amount of cells in the composition is dependent on the relative representation of the T cells with the desired specificity, on the age and weight of the recipient, on the severity of the targeted condition and on the immunogenicity of the targeted Ags. These amount of cells can be as low as approximately 10³/kg, preferably 5×10³/kg; and as high as 10⁷/kg, preferably 10⁸/kg. The number of cells will depend upon the ultimate use for which the composition is intended, as will the type of cells included therein. For example, if cells that are specific for a particular Ag are desired, then the population will contain greater than 70%, generally greater than 80%, 85% and 90-95% of such cells. For uses provided herein, the cells are generally in a volume of a liter or less, can be 500 ml or less, even 250 ml or 100 ml or less. The clinically relevant number of immune cells can be apportioned into multiple infusions that cumulatively equal or exceed the desired total amount of cells.

### Anti-TCR Complex Antibodies:

In some embodiments, the anti-TCR complex antibody is a chimeric antibody. In some embodiments the anti-TCR complex antibody of the present invention is a humanized antibody. In some embodiments, the anti-TCR complex antibody is a human antibody. In some embodiments, the anti-TCR complex antibody is a single chain antibody.

In some embodiments, the anti-TCR complex antibody comprises human heavy chain constant regions sequences but will not induce antibody dependent cellular cytotoxicity (ADCC). In some embodiments, the antibody of the present invention does not comprise an Fc domain capable of substantially binding to a FcgRIIIA (CD16) polypeptide. In some embodiments, the antibody of the present invention lacks an Fc domain (e.g. lacks a CH2 and/or CH3 domain) or comprises an Fc domain of IgG2 or IgG4 isotype. In some embodiments, the antibody of the present invention consists of or comprises a Fab, Fab′, Fab′-SH, F(ab′) 2, Fv, a diabody, single-chain antibody fragment, or a multispecific antibody comprising multiple different antibody fragments. In some embodiments, the antibody of the present invention is not linked to a toxic moiety. In some embodiments, one or more amino acids selected from amino acid residues can be replaced with a different amino acid residue such that the antibody has altered C2q binding and/or reduced or abolished complement dependent cytotoxicity (CDC). This approach is described in further detail in U.S. Pat. No. 6,194,551 by ldusogie et al.

In some embodiments, the anti-TCR complex antibody is selected from the group consisting of TOL101 monoclonal antibodies, TOL101 chimeric antibodies, TOL101 humanized antibodies or variants thereof as described in WO2013067517.

In some embodiments, the anti-TCR complex antibody is an anti-CD3 antibody. A number of anti-CD3 antibodies are known in the art, including but not limited to, OKT3 (muromonab/Orthoclone OKT3™, Ortho Biotech, Raritan, NJ; U.S. Pat. No. 4,361,549); hOKT3Y1 (teplizumab) (MGA031) (Herold et al., NEJM 346 (22): 1692-1698 (2002); Foralumab” and/or “28F11, TRX4 (otelixizumab); HuM291 (Nuvion™, Protein Design Labs, Fremont, CA); gOKT3-5 (Alegre et al., J. Immunol. 148 (11): 3461-8 (1992); 1F4 (Tanaka et al., J. Immunol. 142:2791-2795 (1989)); G4.18 (Nicolls et al., Transplantation 55:459-468 (1993)); 145-2C11 (Davignon et al., J. Immunol. 141 (6): 1848-54 (1988)); and as described in Frenken et al., Transplantation 51 (4): 881-7 (1991); U. S. Pat. Nos. 6,491,9116, 6,406,696, and 6,143,297).

In some embodiments, the anti-CD3 antibody of the present invention comprises a VH and a VL domain selected from Table A.

TABLE A
Examples of anti-CD3 antibodies:

| Name | VH domain | VL domain |
|---|---|---|
| foralumab | QVQLVESGGGVVQPGRSLRLSCAASGFK | EIVLTQSPATLSLSPGERATLSCRASQS |
| | FSGYGMHWVRQAPGKGLEWVAVIWYDGS | VSSYLAWYQQKPGQAPRLLIYDASNRAT |
| | KKYYVDSVKGRFTISRDNSKNTLYLQMN | GIPARFSGSGSGTDFTLTISSLEPEDFA |
| | SLRAEDTAVYYCARQMGYWHFDLWGRGT | VYYCQQRSNWPPLTFGGGTKVEIK (SEQ |
| | LVTVSS (SEQ ID NO: 3) | ID NO : 4) |
| muromonab | QVQLQQSGAELARPGASVKMSCKASGYT | QIVLTQSPAIMSASPGEKVTMTCSASSS |
| | FTRYTMHWVKQRPGQGLEWIGYINPSRG | VSYMNWYQQKSGTSPKRWIYDTSKLASG |
| | YTNYNQKFKDKATLTTDKSSSTAYMQLS | VPAHFRGSGSGTSYSLTISGMEAEDAAT |
| | SLTSEDSAVYYCARYYDDHYCLDYWGQG | YYCQQWSSNPFTFGSGTKLEIK (SEQ |
| | TTLTVSS (SEQ ID NO: 5) | ID NO : 6) |
| otelixizumab | EVQLLESGGGLVQPGGSLRLSCAASGFT | DIQLTQPNSVSTSLGSTVKLSCTLSSGN |
| | FSSFPMAWVRQAPGKGLEWVSTISTSGG | IENNYVHWYQLYEGRSPTTMIYDDDKRP |
| | RTYYRDSVKGRFTISRDNSKNTLYLQMN | DGVPDRFSGSIDRSSNSAFLTIHNVAIE |
| | SLRAEDTAVYYCAKFRQYSGGFDYWGQG | DEAIYFCHSYVSSFNVFGGGTKLTVL |
| | TLVTVSS (SEQ ID NO: 7) | (SEQ ID NO: 8) |
| teplizumab | QVQLVQSGGGVVQPGRSLRLSCKASGYT | DIQMTQSPSSLSASVGDRVTITCSASSS |
| | FTRYTMHWVRQAPGKGLEWIGYINPSRG | VSYMNWYQQTPGKAPKRWIYDTSKLASG |
| | YTNYNQKVKDRFTISRDNSKNTAFLQMD | VPSRFSGSGSGTDYTFTISSLQPEDIAT |
| | SLRPEDTGVYFCARYYDDHYCLDYWGQG | YYCQQWSSNPFTFGQGTKLQIT (SEQ |
| | TPVTVSS (SEQ ID NO: 9) | ID NO: 10) |
| visilizumab | QVQLVQSGAEVKKPGASVKVSCKASGYT | DIQMTQSPSSLSASVGDRVTITCSASSS |
| | FISYTMHWVRQAPGQGLEWMGYINPRSG | VSYMNWYQQKPGKAPKRLIYDTSKLASG |
| | YTHYNQKLKDKATLTADKSASTAYMELS | VPSRFSGSGSGTDFTLTISSLQPEDFAT |
| | SLRSEDTAVYYCARSAYYDYDGFAYWGQ | YYCQQWSSNPPTFGGGTKVEIK (SEQ |
| | GTLVTVSS (SEQ ID NO: 11) | ID NO: 12) |

In some embodiments, the anti-CD3 antibody of the present invention is muromonab having a light chain as set forth in SEQ ID NO:13 and a heavy chain as set forth in SEQ ID NO: 14.

SEQ ID NO: 13
>Muromonab-CD3 light chain
QIVLTQSPAIMSASPGEKVTMTCSASSSVSYMNWYQQKSGTSPKRWIYDT
SKLASGVPAHFRGSGSGTSYSLTISGMEAEDAATYYCQQWSSNPFTFGSG
TKLEINRADTAPTVSIFPPSSEQLTSGGASVVCFLNNFYPKDINVKWKID
GSERQNGVLNSWTDQDSKDSTYSMSSTLTLTKDEYERHNSYTCEATHKTS
TSPIVKSFNRNEC
SEQ ID NO: 14
>Muromonab-CD3 heavy chain
QVQLQQSGAELARPGASVKMSCKASGYTFTRYTMHWVKQRPGQGLEWIGY
INPSRGYTNYNQKFKDKATLTTDKSSSTAYMQLSSLTSEDSAVYYCARYY
DDHYCLDYWGQGTTLTVSSAKTTAPSVYPLAPVCGGTTGSSVTLGCLVKG
YFPEPVTLTWNSGSLSSGVHTFPAVLQSDLYTLSSSVTVISSTWPSQSIT
CNVAHPASSTKVDKKIEPRPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK

In some embodiments, the anti-CD3 antibody of the present invention is teplizumab having a light chain as set forth in SEQ ID NO:15 and a heavy chain as set forth in SEQ ID NO:16.

SEQ ID NO: 15
>8869_L|teplizumab|Humanized||L-KAPPA (V-KAPPA(1-
105) + C-KAPPA(107-213))|||||||213||||MW 23304.7|
MW 23304.7|
DIQMTQSPSSLSASVGDRVTITCSASSSVSYMNWYQQTPGKAPKRWIYDT
SKLASGVPSRFSGSGSGTDYTFTISSLQPEDIATYYCQQWSSNPFTFGQG
TKLQITRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC
SEQ ID NO: 16
>8869_H|teplizumab|Humanized||H-GAMMA-1 (VH (1-
119)+ CH1(120-217) + HINGE-REGION (218-232) +
CH2(233-342) + CH3(343-449))|||||||449||||
MW 49611.9|MW 49611.9|
QVQLVQSGGGVVQPGRSLRLSCKASGYTFTRYTMHWVRQAPGKGLEWIGY
INPSRGYTNYNQKVKDRFTISRDNSKNTAFLQMDSLRPEDTGVYFCARYY
DDHYCLDYWGQGTPVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPEAAGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK

Typically, the anti-TCR complex antibody of the present invention is administered to the subject in the form of pharmaceutical composition. The pharmaceutical composition may be produced by those of skill, employing accepted principles of treatment. Such principles are known in the art, and are set forth, for example, in Braunwald et al., eds., Harrison's Principles of Internal Medicine, 19th Ed., McGraw-Hill publisher, New York, N.Y. (2015), which is incorporated by reference herein. The pharmaceutical composition may be administered by any means that achieve their intended purpose. For example, administration may be by parenteral, subcutaneous, intravenous, intradermal, intramuscular, intraperitoneal, transdermal, or buccal routes. The pharmaceutical compositions may be administered parenterally by bolus injection or by gradual perfusion over time. The pharmaceutical compositions typically comprises suitable pharmaceutically acceptable carriers comprising excipients and auxiliaries which may facilitate processing of the active compounds into preparations which can be used pharmaceutically. The pharmaceutical compositions may contain from about 0.001 to about 99 percent, or from about 0.01 to about 95 percent of active compound(s), together with the excipient.

The invention will be further illustrated by the following figures and examples. However, these examples and figures should not be interpreted in any way as limiting the scope of the present invention.

### FIGURES

FIG. 1 Schematic experimental design: Eight- to 12-week-old male NSG mice were conditioned with busulfan on Days −3 and −2 before IV injection of 5.10⁶ HLA-A2+ PBMCs through the tail vein, on Day −1. The following Day (Day0), a mix of 2,5.10⁶ CD3-sufficient and CD3-deficient of CAR-T cells, at a 1:1 ratio, was administered through retroorbital injection. CAR-T cells were produced over a two-week protocol as detailed in the methods. Briefly, the final cell product included 50% GFP+CD3-deficient CAR-T and 50% mCherry/Luc+−CD3-sufficient CAR-T. Mice were split in two groups of 8 individuals, treated intravenously either with 5 μg/day otelixizumab (non-mitogenic anti-human CD3) or with saline solution, 4 consecutive days, starting Day3 (after Day3 blood draw and in vivo imaging). A phenotypic analysis of circulating cells was performed with flow-cytometry on Days 3, 7 and 13. Bioluminescence imaging (BLI) was performed on Day 3, 6 and 12. Half of the mice of each group was taken down at Day7, the remaining half at Dayl3.

FIG. 2. Representative flow cytometry plots in the blood at different time points. The respective proportion of murine and human cells is depicted on the left panel. The respective proportion of CD3-sufficient CAR T (mCherry-positive), CD3-deficient CAR-T (GFP-positive) and HLA A2+ human cells (mCherry and GFP double negative) is depicted on the right panel.

FIG. 3. Graphs showing human chimerism frequencies and CAR T subpopulations ratios at different time points and in different tissues (blood, spleen, bone marrow).

FIG. 4. In vivo imaging. The signal after Luciferin injection was acquired using an IVIS SpectrumCT in order to provide a semi-quantitative assessment of luciferase-expressing CD3-sufficient CAR-T cells. BLI analysis was performed at day 3, 6 and 12 after CAR-T cell injection. A region of interest (ROI) was applied (left panels), and the signal emitted by bioluminescent cells is shown using a flux unit (photon/second) in right panels.

FIG. 5. CAR-Treg stability and activation upon stimulation according to TCR gene editing. A. Experimental design for TCR-deficient CAR-Treg manufacturing: naïve Tregs were FACS sorted from enriched CD4+ cells and activated the next day. Activated Tregs were transduced with a CAR-encoding lentiviral vector on day 2 and subsequently underwent CRISPR-Cas9-mediated TRAC disruption two days later. CAR Tregs were FACS sorted on day 7 and reactivated through the CAR on day 9 of culture. B. CAR Treg stability was assessed 2 days after CAR stimulation according to the presence (left panel) or not (right panel) of the TCR/CD3 complex. C. Representative flow cytometry histograms depicting activation markers assessed on CD3/TCR-sufficient or deficient CAR-Tregs either stimulated (down panel) or not (upper panel) through the CAR.

### EXAMPLE

### Methods

### HLA A2-Targeted CAR Design

The CAR was designed as described in Lamarthée et al. Nat Commun 2021. Briefly the selected scFv was fused to a stalk region from the human CD8α hinge, the transmembrane domain from human CD8, the CD28 or 4-1BB CSD, and the human CD3ζ signaling domain. EGFRt was cloned downstream of T2A at the second gene position to serve as a reporter

### HLA A2-Targeted CAR-T Cell Generation

The CAR-T cell was generated as described in Lamarthée et al. Nat Commun 2021. Briefly, healthy donor PBMCs were obtained from the Etablissement Fran ais du Sang. PBMCs were typed based on the expression or absence of HLA-A2/A28 molecules, as assessed by anti-HLA-A2/A28 antibody (OneLambda) staining evaluated by flow cytometry using the BD LSRFortessa™ X-20 analyzer. CD4+ T cells were then enriched from HLA-A2-donor PBMCs using an EasySep CD4+Enrichment kit (Stem Cell®). Two days after activation, T cells were transduced with the HLA A2-targeted CAR construct at a multiplicity of infection (MOI) of 30 viruses per cell. Prostaglandin E2 (PGE2) (Cayman Chemical, 10 μM) and a transduction adjuvant (Lentiboost, Sirion Biotech, 0.25 mg/mL) were added for a 6 h of transduction time.

### Purification of Human Tregs

Healthy donor PBMCs were obtained from the Etablissement Français du Sang. PBMCs were typed based on the expression or absence of HLA-A2/A28 molecules, as assessed by anti-HLA-A2/A28 antibody (OneLambda) staining evaluated by flow cytometry using the BD LSRFortessa™ X-20 analyzer. CD4+ T cells were enriched from HLA-A2-donor PBMCs using an EasySep CD4+Enrichment kit (Stem Cell®). Naive regulatory T cells (nTregs), which were defined as CD4+CD25^{Hi} CD127−CD45RA+CD45RO−, were sorted using a FACS Aria II (BD Biosciences). In parallel, we sorted CD4+CD45RA+CD25− Tconv cells from the same donor as controls. The sorting purity was checked with flow cytometry and always demonstrated a purity greater than 98%. Sorted T cells were stimulated with Dynabeads™ Human T-Activator CD3/CD28 (Thermo Fisher Scientific) (ratio 1:1) in X-VIVOR 20 medium containing 10% human serum AB (Biowest) and 1000 UI/mL human IL-2 (Proleukin, Novartis). HLA-typed CD3-depleted splenocytes were isolated from spleens collected from deceased organ donors through collaboration with the Regional Histocompatibility Laboratory and National Biomedicine Agency.

### HLA A2-Targeted CAR-Treg Cell Generation

The CAR-T cell was generated as described in Lamarthée et al. Nat Commun 2021. Two days after activation, Tregs were transduced with the different CAR constructs at a multiplicity of infection (MOI) of 30 viruses per cell. Prostaglandin E2 (PGE2) (Cayman Chemical, 10 μM) and a transduction adjuvant (Lentiboost, Sirion Biotech, 0.25 mg/mL) were added for a 6 h of transduction time. On day 7 posttransduction, CD4+EGFRt+ cells were sorted using a FACS Aria II and then restimulated with anti-CD3/CD28 beads. T cells were expanded and cultured in complete medium supplemented with IL-2 (1000 UI/mL) for 7 to 10 days for in vitro experiments. Three Treg cultures (separate experiments) were interrupted because the FOXP3+ frequency among expanded untransduced Tregs had dropped below 50% at Day 10. These experiments were considered as failures, regardless the cause (sorting, donor, IL-2, . . . ), and excluded from further analysis.

### TRAC Deletion Using CRISPR/Cas9 Genome Editing

The TRAC deletion in the CAR-T cells was performed as described in Lamarthée et al. Nat Commun 2021. Briefly gRNA sites in human TRAC exonic loci were identified using the online optimized design software at http://crispor.tefor.net/72. The highest scoring gRNA, which had no off-target sequences with perfect matches in the human genome, the best predicted efficiency and the nearest coding off-target exonic sites containing at least three mismatched nucleotides was selected and purchased from Thermo Fisher (TrueGuide Synthetic sgRNA, Invitrogen). The TRAC CRISPR RNA (crRNA)-targeting sequences included CTCTCAGCTGGTACACGGCA GGG (SEQ ID NO:17).

### Nucleofection

The nucleofection was performed as described in Lamarthée et al. Nat Commun 2021. Briefly, on the indicated culture day, cells were resuspended in a Cas9 nuclease/gRNA/nucleofection reagent complex using the P3 Primary Cell 4DNucleofector X Kit (Lonza) and underwent nucleofection in a 4D-Nucleofector™ Core Unit+4D-Nucleofector™ X Unit (Lonza) using the EO 115 program according to the manufacturer's instructions. Cells were then split into prewarmed culture medium at 10⁶ cells/mL with IL-2 (1000 UI/mL) and incubated at 37° C. and 5% CO2 for 24 h before removing stimulation anti-CD3/CD28 beads.

### XenoGVHD

All appropriate procedures were performed in the animal facility (registration number A75-15-34) and followed to ensure animal welfare. Eight- to 12-week-old male NSG mice (bred in house or purchased from Charles River) were intraperitoneally injected with 25 mg/kg busulfan (Merck) on days −2 and −1 before injection of 5× 10⁶ HLA-A2+ PBMCs into the tail vein with or without 5×10⁶ CAR-Tregs injected retroorbitally via the venous sinus. Saline-injected mice served as controls. CAR-Tregs were generated from three different healthy donors. GVHD was scored based on weight, hunching, fur properties, diarrhea and skin integrity, with 0 to 1 point per category as previously described⁷¹. On the indicated days and after isoflurane anesthesia supplemented with tetracaine analgesia, peripheral blood from the venous sinus was harvested and centrifuged, and the plasma was collected and frozen before cytokine measurement. Then, the erythrocytes were lysed (RBC lysis buffer, Ozyme), and leukocytes were evaluated by flow-cytometry analysis. When a mouse reached a score of 4, it was sacrificed by cervical dislocation, and the spleen was collected for flow-cytometry analysis, whereas the lungs and liver were harvested for histology. Mouse tissues were fixed in 4% paraformaldehyde and paraffin embedded. Liver and lung sections (4 μm) were stained with hematoxylin and eosin, scanned (Nanozoomer 2.0, Hamamatsu) and blindly assessed by two independent researchers using NDPview software (Hamamatsu).

### Results

The inventors found that CAR-Tregs outperformed polyclonal Tregs in preventing xenogeneic GVHD. They provided a thorough comparison between CD28 and 41BB costimulation endodomains in terms of CAR-Treg proliferation, function, phenotype, metabolism and signaling.

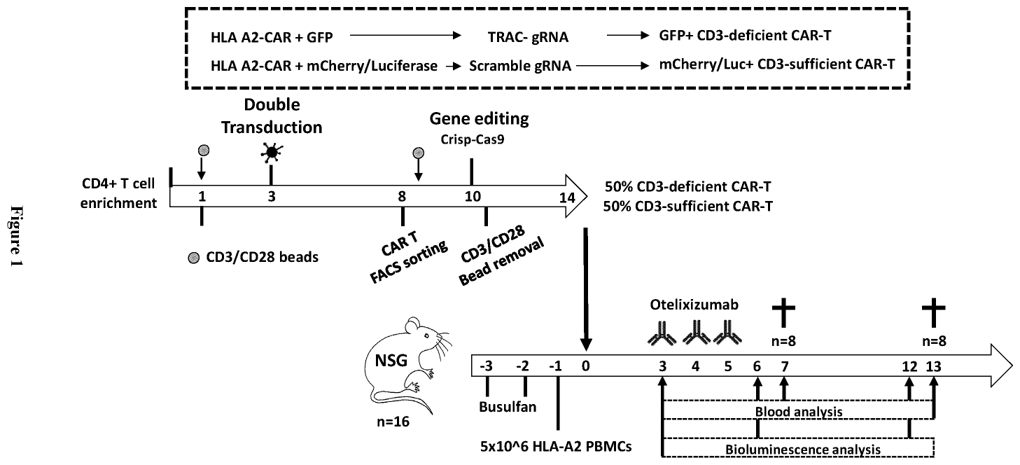

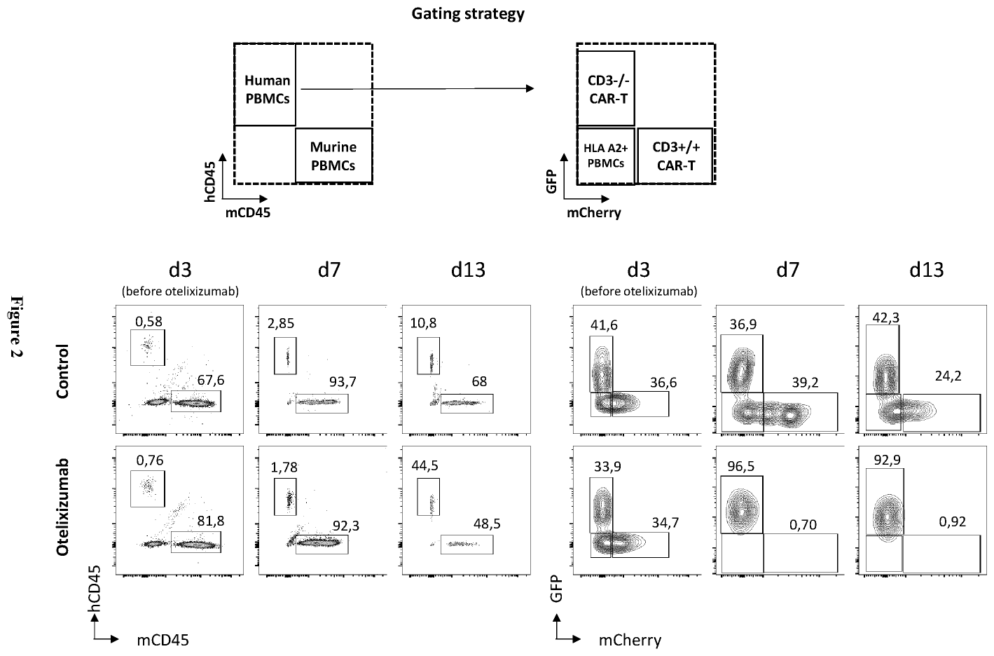

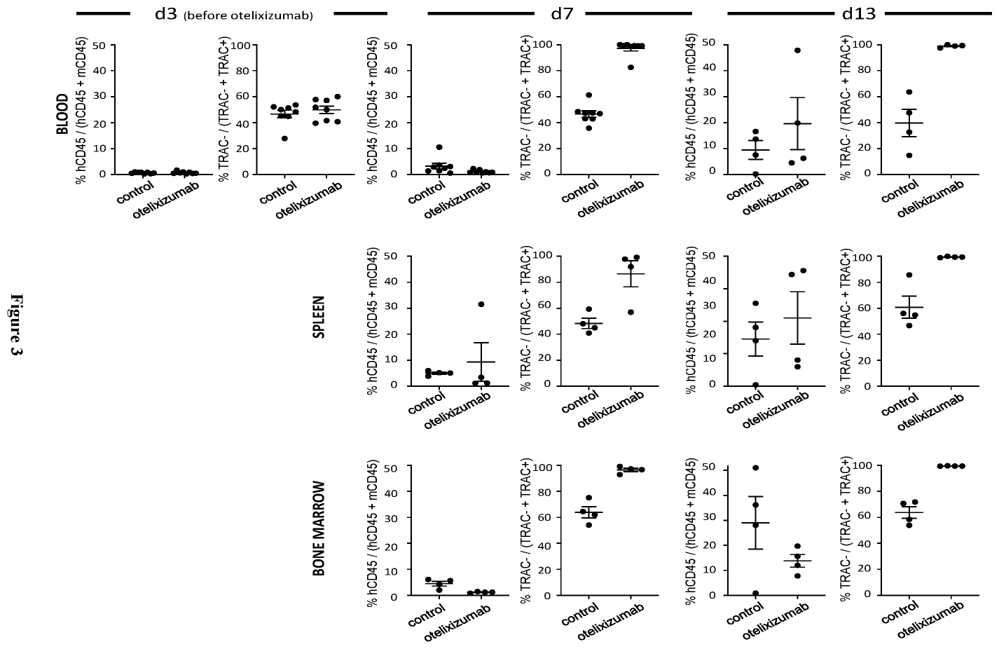

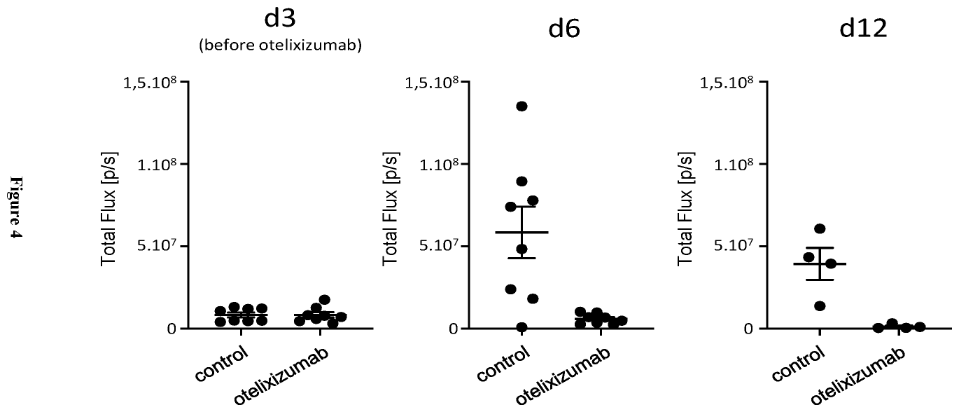

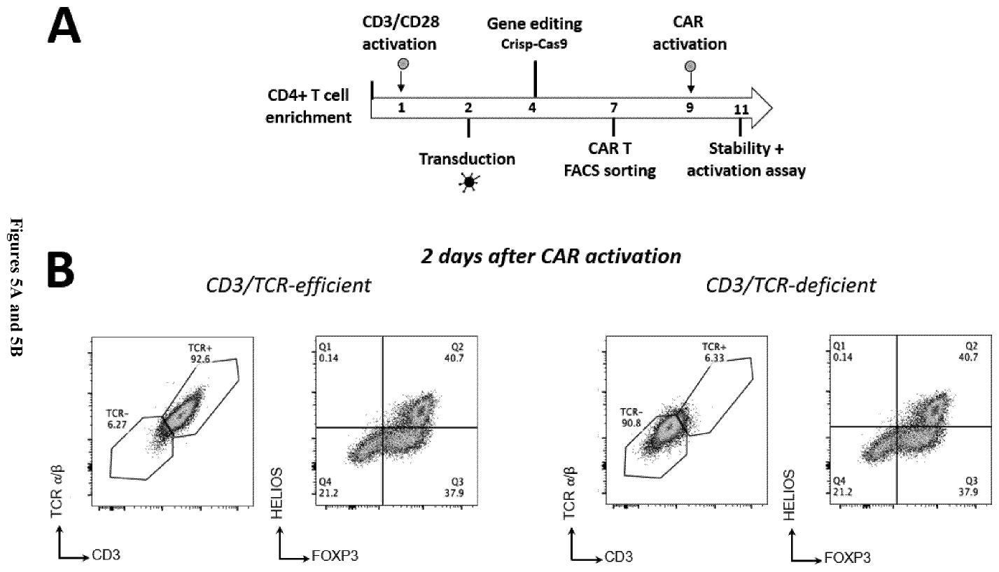

They shortened the in vitro expansion of CAR-Tregs. In addition, they showed that TCR-deficient CAR-T cells were spared by anti-CD3 antibody. Hence, combination of anti-CD3 with TRAC-deficient CAR-T cells provide them with an in vivo life advantage over the TRAC sufficient T cells. They have optimized CD3/TCR complex disruption in CAR-Tregs through Crispr-Cas9 gene editing. This finding lays the groundwork for synergistic therapy combining TCR-deficient CAR-Tregs and anti-CD3 antibody to promote immune tolerance. (FIGS. 1 to 4).

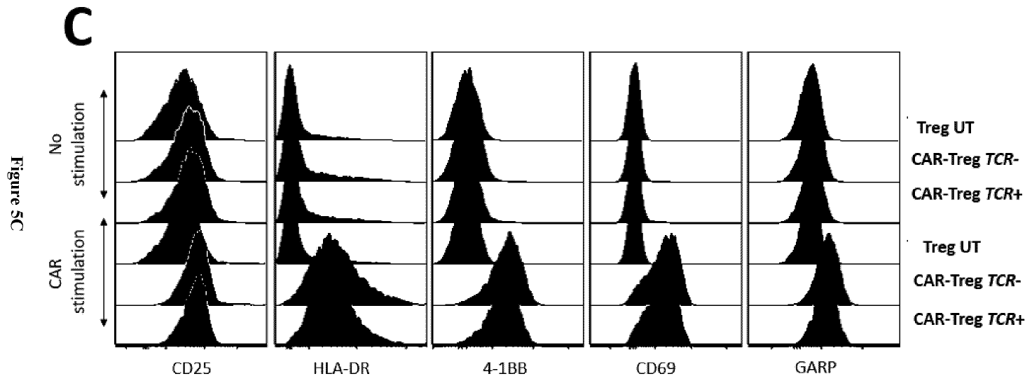

The inventors also showed the ability to stimulate by CAR, a CAR-Treg deficient for the TRAC gene, in the same way as its TRAC-sufficient counterpart. Similarly, they showed that the deletion of the TRAC gene does not negatively impact the stability of the cell (FIGS. 5A to 5C).

### REFERENCES

Throughout this application, various references describe the state of the art to which this invention pertains. The disclosures of these references are hereby incorporated by reference into the present disclosure.

1. A method of inducing tolerance to an antigen of interest in a subject in need thereof comprising administering to the subject a therapeutically effective combination of a population of TCR-deficient CAR-Tregs cells specific for said antigen with an amount of an anti-TCR complex antibody.

2. The method of claim 1 wherein the subject is predisposed or believed to be predisposed to developing, or has already developed or is developing an autoimmune disease.

3. The method of claim 1 wherein the subject is predisposed or believed to be predisposed to developing, or has already developed or is developing an allergy.

4. The method of claim 1 wherein the subject is predisposed or believed to be predisposed to developing, or has already developed or is developing an immune reaction against molecules that are exogenously administered for therapeutic or other purposes.

5. The method of claim 1 wherein the subject is predisposed or believed to be predisposed to developing, or has already developed or is developing an immune reaction against a grafted population of cells, a grafted tissue or a grafted organ.

6. The method of claim 1 wherein the subject suffers from Host-Versus-Graft-Disease (HVGD) and Graft-Versus-Host-Disease (GVHD).

7. The method of claim 1 wherein the antigen is an auto-antigen, an allergen, an allo-antigen or a molecule that is exogenously administered for therapeutic purposes.

8. The method of claim 7 wherein the allo-antigen is HLA-A2.

9. The method of claim 1 wherein the CAR-Tregs cells comprise the CD3ζ signaling domain and the 4-1BB co-stimulatory signaling domain.

10. The method of claim 1 wherein the population of TCR-deficient CAR-Treg cells is obtained by i) introducing a nucleic acid encoding a CAR into a cell and ii) contacting the cell with an endonuclease system so as to repress the expression of TCR complex.

11. The method of claim 10 wherein the population of TCR-deficient CAR-Treg cells is obtained by i) introducing a nucleic acid encoding a CAR into a cell and ii) contacting the cell with a Cas protein and with at least one guide RNA molecules (gRNA) comprising a sequence that targets the TRAC gene, and a sequence which is capable of binding to the Cas protein.

12. The method of claim 1 wherein the anti-TCR complex antibody is a chimeric antibody, a humanized antibody or a human antibody.

13. The method of claim 1 wherein the anti-TCR complex antibody comprises human heavy chain constant regions sequences but does not induce antibody dependent cellular cytotoxicity.

14. The method of claim 1 wherein the anti-TCR complex antibody is an anti-CD3 antibody.

15. The method of claim 14 wherein the anti-CD3 antibody is selected from the group consisting of foralumab, muromonab, otelixizumab, teplizumab, and visilizumab.
